(19) United States
(12) Patent Application Publication
(10) Pub. No.: US 2019/0298166 A1
(43) Pub. Date: Oct. 3, 2019

(54) SYSTEM AND METHOD FOR TESTING VISUAL FIELD

(71) Applicant: Virtual Field, Inc., New York, NY (US)

(72) Inventors: Aaron Henry Smith, New York, NY (US); Mark Harooni, Old Westbury, NY (US); Carl Vincent Block, Carlsbad, CA (US)

(21) Appl. No.: 16/359,046

(22) Filed: Mar. 20, 2019

Related U.S. Application Data
(60) Provisional application No. 62/689,131, filed on Jun. 23, 2018.
(60) Provisional application No. 62/648,749, filed on Mar. 27, 2018.

(51) Int. Cl. A61B 3/024; G06T 19/00; G06F 3/0482; A61B 3/06; A61B 3/00

(52) U.S. Cl. CPC A61B 3/024; G06T 19/006; A61B 3/0091; A61B 3/066; A61B 3/0058; G06F 3/0482

(57) ABSTRACT

A virtual reality system can perform a visual field test for detecting an ocular disorder. The system can include a display unit and a virtual reality headset. The virtual reality headset can be in wireless communication with the display unit. The virtual reality headset can include a display screen configured to present a test scene to a patient to thereby detect the ocular disorder.

### CROSS-REFERENCE TO RELATED APPLICATIONS

This application claims priority to U.S. Provisional Application No. 62/689,131, filed on Jun. 23, 2018, titled “System and Method for Mapping the Visual Field and Training New PRLS”, and claims priority to U.S. Provisional Application No. 62/648,749, filed on Mar. 27, 2018, titled “System and Method for Testing Visual Field”, the entireties of each of which are incorporated by reference herein.

### BACKGROUND

Visual field testing can be used for finding scotomas (blind spots) and/or peripheral vision loss, among other symptoms indicating ocular disorders. Scotomas and/or peripheral visional loss, among other symptoms can indicate glaucoma, and/or other ocular diseases. A patient can be tested frequently on the same machine over a long period of time to detect gradual changes in their vision. In each of these tests, the patient fixates on a center point while a stimulus is moved or flashed at another position in the patient's visual field. Testing procedures can be inefficient, inaccurate, slow, and/or require medical professionals to administer the entire test, thereby increasing costs and time associated with testing a patient's visual field.

### SUMMARY

Systems, methods, and articles of manufacture, including computer program products, are provided for detecting ocular disorders and other disorders or diseases, including, but not limited to glaucoma, amblyopia, diabetes (e.g., diabetic retinopathy), brain tumors, strokes, retinal scars, retinal degeneration, parietal lobe issues, frontal lobe issues, optic tract issues, cataracts, optic neuropathy, occipital strokes, junctal strokes, tumor behind the optic nerve, and/or the like. In one aspect, there is provided a system. The system may include at least one data processor and at least one memory. The at least one memory may store instructions that result in operations when executed by the at least one data processor.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to web application user interfaces, it should be readily understood that such features are not intended to be limiting. The claims that follow this disclosure are intended to define the scope of the protected subject matter.

In some implementations, there may be provided a method of performing a visual field test to detect an ocular disorder via a virtual reality headset. The method may include presenting, via a display screen by a virtual reality engine of the virtual reality headset, the test scene. The test scene may include a background color and a fixation point. The method may also include testing, automatically by the virtual reality engine via the display screen, a blind spot of the patient. The testing may include presenting, by the virtual reality engine on the display screen, a stimulus at a randomly assigned location from a queue of locations. The stimulus may have a stimulus color. The method may further include receiving, by the virtual reality engine, a response from the patient based on the presented stimulus.

In some variations, one or more of the features disclosed herein including the following features can optionally be included in any feasible combination. The method may include receiving, via a user interface of a display unit in communication with the virtual reality headset, one or more of patient information, the stimulus color, and the background color. In some variations, the method may include assigning, by the virtual reality engine, a timestamp to the received response from the patient. The method may include storing, by the virtual reality engine, in the at least one memory, the randomly assigned location of the stimulus and the assigned timestamp.

In some variations, the method may include transmitting, by the virtual reality engine, the stored randomly assigned location of the stimulus and the assigned timestamp to a display unit in communication with the virtual reality headset. The method may further include testing, automatically via the display screen, a static or kinetic perimeter of the patient. The testing may include presenting the stimulus having the stimulus color on the display screen.

In some variations, the response from the patient includes an invalid response. In some variations, the method further includes comparing, by the virtual reality engine, a number of received invalid responses to a predetermined threshold. The method may further include restarting, by the virtual reality engine, the testing, such as when the number of received invalid responses is greater than or equal to the predetermined threshold.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A depicts a system diagram illustrating a visual field analyzer system, in accordance with some example embodiments;

FIG. 1B depicts a system diagram illustrating a visual field analyzer system, in accordance with some example embodiments;

FIG. 2 illustrates an example user interface, in accordance with some example embodiments;

FIG. 3 illustrates an example user interface, in accordance with some example embodiments;

FIG. 4 illustrates an example user interface, in accordance with some example embodiments;

FIG. 5 illustrates an example display scene, in accordance with some example embodiments;

FIG. 6 illustrates an example display scene, in accordance with some example embodiments;

FIG. 7 depicts a flowchart illustrating a process for performing a visual field exam, in accordance with some example embodiments;

FIG. 8 depicts a flowchart illustrating a process for performing a visual field exam, in accordance with some example embodiments;

FIG. 9 depicts a flowchart illustrating a process for capturing measurements during a visual field exam, in accordance with some example embodiments;

FIG. 10 depicts an example architecture for performing a visual field exam, in accordance with some example embodiments;

FIG. 11 depicts a flowchart illustrating a process for implementing luminance and/or contrast during a visual field exam, in accordance with some example embodiments;

FIG. 12 illustrates an a visual field schematic representing an example field of vision, in accordance with some example embodiments;

FIGS. 13A-13B illustrate an example display scene, in accordance with some example embodiments;

FIGS. 14A-14B illustrate an example display scene, in accordance with some example embodiments;

FIGS. 15A-15D illustrate an example display scene, in accordance with some example embodiments;

FIG. 16 depicts a flowchart illustrating a process for mapping a scotoma in the visual field, in accordance with some example embodiments;

FIGS. 17A-17C illustrate a visual field schematic representing an example field of vision, in accordance with some example embodiments;

FIG. 18 depicts a flowchart illustrating a process for determining an eccentric preferred retinal locus, in accordance with some example embodiments;

FIG. 19 depicts a flowchart illustrating a process for training a patient to use an eccentric preferred retinal locus, in accordance with some example embodiments;

FIG. 20 illustrates an visual field testing system, in accordance with some example embodiments; and

FIG. 21 illustrates an visual field testing system, in accordance with some example embodiments.

When practical, similar reference numbers denote similar structures, features, or elements.

### DETAILED DESCRIPTION

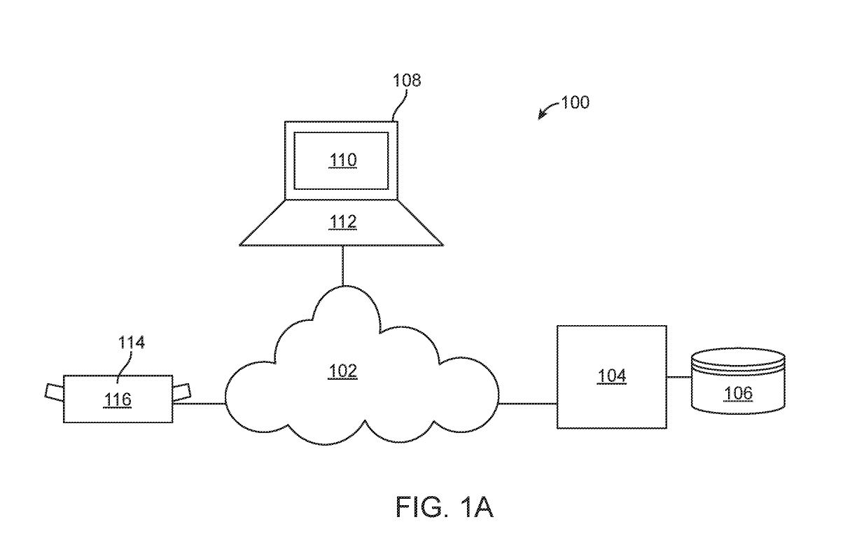

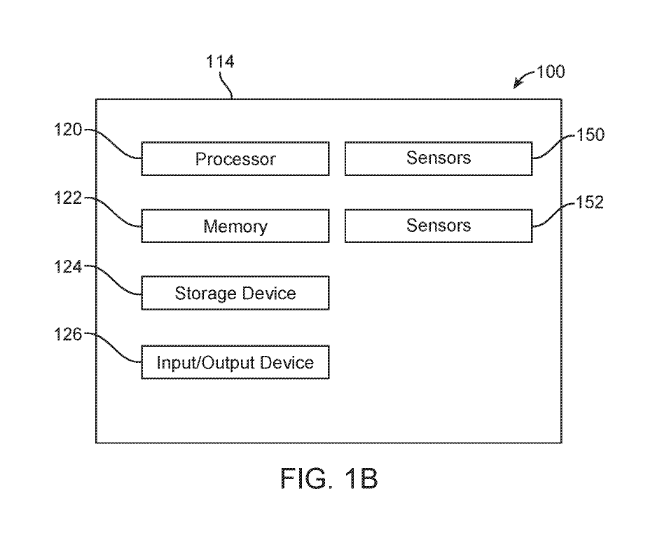

FIG. 1A depicts a system diagram illustrating a visual field analyzer system 100, in accordance with some example embodiments. The system 100 can include a controller 104, a virtual reality headset 114 having at least one display screen and a virtual reality engine 116, and/or a display unit 108 having a user interface 110 and an administrative engine 112, among other features. Although various features described herein, such as the virtual reality headset 114 and the virtual reality engine 116 are described with respect to a virtual reality system, the features may similarly be implemented in an augmented reality and/or a mixed reality system.

As shown in FIG. 1, the controller 104, the virtual reality headset 114, and/or the display unit 108 may be communicatively coupled via a network 102. The network 102 may be any wired and/or wireless network including, for example, a public land mobile network (PLMN), a wide area network (WAN), a local area network (LAN), a virtual local area network (VLAN), the Internet, and/or the like. Any data received at the controller 104 may be evaluated by the controller 104 in real time and/or stored at a database 106 coupled with the controller 104 for evaluation at a later time.

FIG. 1B depicts a diagram illustrating a computing system, such as the visual field analyzer system 100 consistent with implementations of the current subject matter. The system 100 can be used to implement the display unit 108, the controller 104, and/or the virtual reality headset 114, any combination thereof, and/or any components therein.

As shown in FIG. 1B, the system 100 can include a processor 120, a memory 122, a storage device 124, and input/output devices 126. The processor 120, the memory 122, the storage device 124, and/or the input/output devices 126 can be interconnected via a wired and/or wireless connection. The processor 120 is capable of processing instructions for execution within the system 100. Such executed instructions can implement one or more components of the system 100. In some example embodiments, the processor 120 can be a single-threaded processor. Alternatively, the processor 120 can be a multi-threaded processor. The processor 120 can be capable of processing instructions stored in the memory 122 and/or on the storage device 124 to display graphical information for a user interface provided via the input/output device 126, such as the virtual reality headset 114, the display unit 108, and/or another component.

The memory 122 can be a computer readable medium such as volatile or non-volatile that stores information within the system 100. The memory 122 can store data structures representing configuration object databases, for example. The storage device 124 can be capable of providing persistent storage for the system 100. The storage device 124 can be a floppy disk device, a hard disk device, an optical disk device, or a tape device, or other suitable persistent storage means. The input/output device 126 can provide input/output operations for the system 100. In some example implementations, the input/output device 126 includes a keyboard and/or pointing device. In various implementations, the input/output device 126 includes a display unit for displaying graphical user interfaces.

According to some example implementations, the input/output device 126 can provide input/output operations for a network device. For example, the input/output device 126 can include Ethernet ports or other networking ports to communicate with one or more wired and/or wireless networks (e.g., a local area network (LAN), a wide area network (WAN), the Internet).

In some example embodiments, the system 100 can be used to execute various interactive computer software applications that can be used for organization, analysis and/or storage of data in various formats. Alternatively, the system 100 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 126. The user interface can be generated and presented to a user by the system 100 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### Visual Field Analyzer System

There are typically two primary methods of visual field testing: (1) kinetic and (2) static. Kinetic testing is typically manually operated with a display screen, a large piece of paper, and a flashlight. The physician can move the light or another stimulus toward the center of the paper from the perimeter of the paper. The patient can indicate when the stimulus is in their visual field by tapping on a table, at which point the doctor places a mark on the paper. In some implementations of kinetic testing, progressively less intense (e.g., less bright) stimuli can be used to create rings of the patient's visual field on the paper by moving the stimulus to different locations. The resulting rings of the patient's visual field is compared to a baseline result. The patient may have glaucoma, or another ocular disorder if the patient's visual field deviates from the baseline result. The typical kinetic test is typically very difficult to perform, and the results may be inaccurate. The kinetic test can undesirably rely on the physician to administer the test accurately and to mark the paper in the correct location. Such results may produce false readings and can lead to an incorrect determination of glaucoma, no-glaucoma, or other ocular disorder.

Unlike a kinetic test, tests using a static stimulus (e.g., static perimetry) typically include flashes of light at individual points. The individual points are typically positioned 6° apart, in a small basin. The test may be automated, and the patient can click on a trigger attached to the machine if the patient sees the stimulus. The test is typically performed by testing one of the patient's eyes at a time. The eye that is not being tested is typically patched or otherwise forcibly held shut, which in some instances can negatively impact test results.

A typical analyzer machine, such as a Humphrey Field Analyzer, can be used to administer the test. The machine can have in-built eye tracking capabilities. The test can be used to detect the patient's natural blind spot (e.g., the place in the patient's field of vision where the patient cannot see anything). The blind spot is typically positioned 1.5° below center and 12-15° temporally (e.g., toward the temple on the same side as the eye).

In the static test, false positives may occur if the patient clicks when no light stimulus has been presented. Fixation losses can be recorded if the patient clicks when stimuli are presented in the blind spot where the patient should not be able to see them unless the patient is not fixated on the center point. Such techniques may be referred to as the Heijil-Krakau technique. False negatives may be detected by flashing bright stimuli that the patient should be able to see at points where the patient has already been found to see a stimulus. If the patient cannot see the stimulus, the patient may have lost focus and may no longer be taking the test. When the test is completed, a report can be generated with two pages, for example. Each page can include a report on each eye. The report can illustrate where vision is weak and/or not present (e.g., a scotoma, or blind spot). Typical tests may require skilled professionals to administer the test, increasing time, cost, and inaccuracies. The physician typically must monitor the machine being use to administer the test.

The status perimetry test can use a Fast Threshold test. The Fast Threshold test is a method used for testing visual field loss—typically in testing for glaucoma, among other ocular disorders. The Fast Threshold test can optimize the determination of perimetry thresholds by continuously estimating the expected threshold based certain information, such as the patient's age and/or neighboring thresholds.

In some implementations, the visual field analyzer system 100 can be used to perform a visual field exam. The visual field analyzer system 100, as shown in FIGS. 1A and 1B, can include the virtual reality engine 116 and/or the administrative engine 112. As mentioned above, the display unit 108 can include the administrative engine 112. In some implementations, the virtual reality headset 114 can include the administrative engine 112, the display unit 108, the controller 104 and/or the database 106, among other components. In some implementations, the virtual reality headset 114 and the display unit 108, the controller 104 and/or the database 106, among other components, are separately connected. The administrative engine 112 can be used by a user, such as a technician, medical professional, or other non-professional via the user interface 110 to start, pause, restart, and/or stop the visual field test, among other features. The administrative engine 112 can be implemented in the virtual reality headset 114 and/or the display unit 108 to automatically start, pause, restart, and/or stop the visual field exam, among other features. In some implementations, the administrative engine 112 can be used by the user to analyze, export, and/or print various configurations of the results of the visual field exam.

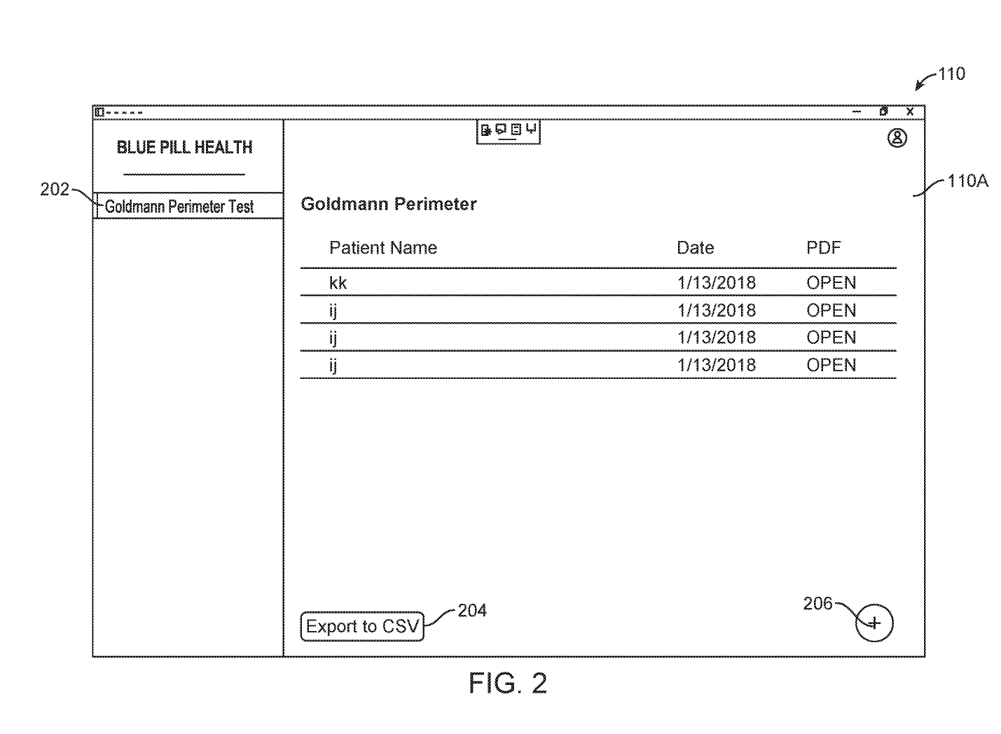

FIG. 2 illustrates an example user interface 110 according to implementations of the current subject matter. The user interface 110 illustrated in FIG. 2 shows an example results display 110A. The results display 110A can list certain exam types 202. In some implementations, the exam types 202 can be displayed on one side, such as the left side of the results display 110A. The exam types 202 can include the Goldmann Perimeter, Fast Threshold test, and/or other Fast tests, among other types of exam types that can be used for analyzing a patient's visual field to treat certain ocular disorders, as discussed herein.

The results display 110A can display previous exams that have been administered to a particular patient and/or to various patients that have used the virtual reality headset 114. The previous exams can be displayed at a center region of the results display 110A. The results can be exported to a printer, display unit, or other device via wired or wireless connection when an export button 204 displayed on the results display 110A is selected via the user interface 110. In some implementations, a new exam can begin when a new exam button 206 displayed on the results display 110A is selected via the user interface 110. In some implementations, the new exam can begin automatically or after a predetermined time period once the virtual reality headset 114 is properly positioned.

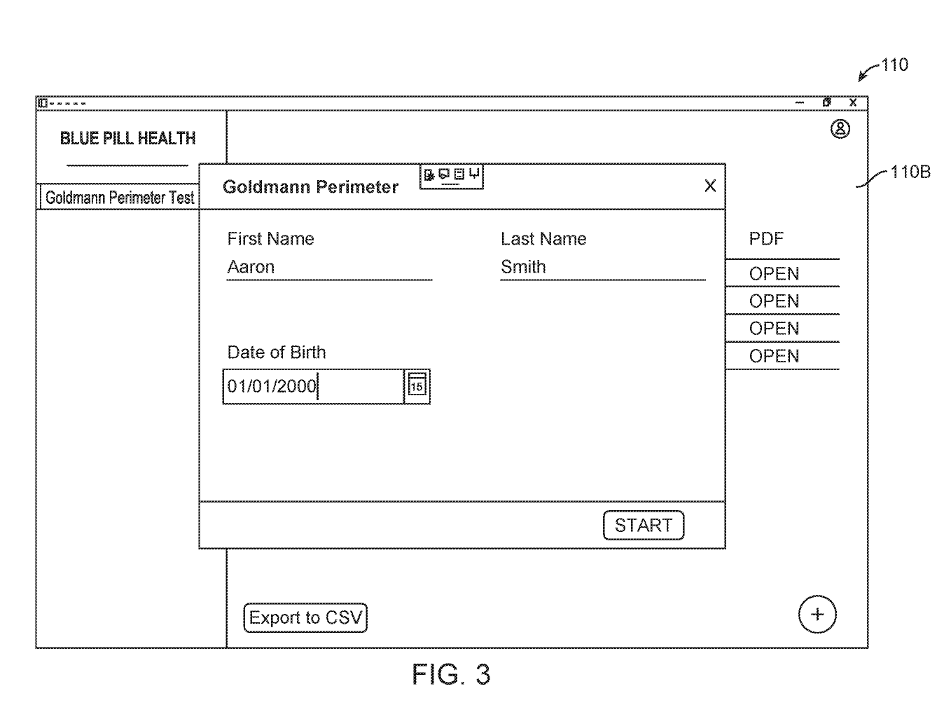

FIG. 3 illustrates an example user interface 110 according to implementations of the current subject matter. The user interface 110 illustrated in FIG. 3 shows an example patient information display 110B. The patient information display 110B can receive information about the patient via the user interface 110. In some implementations, the patient information display 110B can retrieve information about the patient that is stored in the database 106.

In some implementations, the patient information display 110B can display one or more stimulus and/or background color schemes, as described in more detail below. The patient information display 110B can be configured to receive a selection of the one or more stimulus and/or background color schemes. The stimulus and/or background color schemes can be displayed to the patient when the virtual reality headset 114 is worn by the patient. Typically, machines or other platforms used for administering the visual field exam display a while light (e.g., a stimulus) shown in a white basin (e.g., the background). Using typical platforms, it can be very expensive and/or difficult to alter the stimulus and/or background color schemes. The system 100 can desirably allow for the user to quickly and/or easily select a desired stimulus and/or background color scheme. In some implementations, the system 100 can display the selected stimulus and/or background color scheme easily and/or quickly. In some implementations, the system 100 can display the stimulus and/or background color scheme automatically based on the patient's information that is stored in the database 106. In some implementations, a black stimulus and a white background can be selected and/or displayed, among other color combinations.

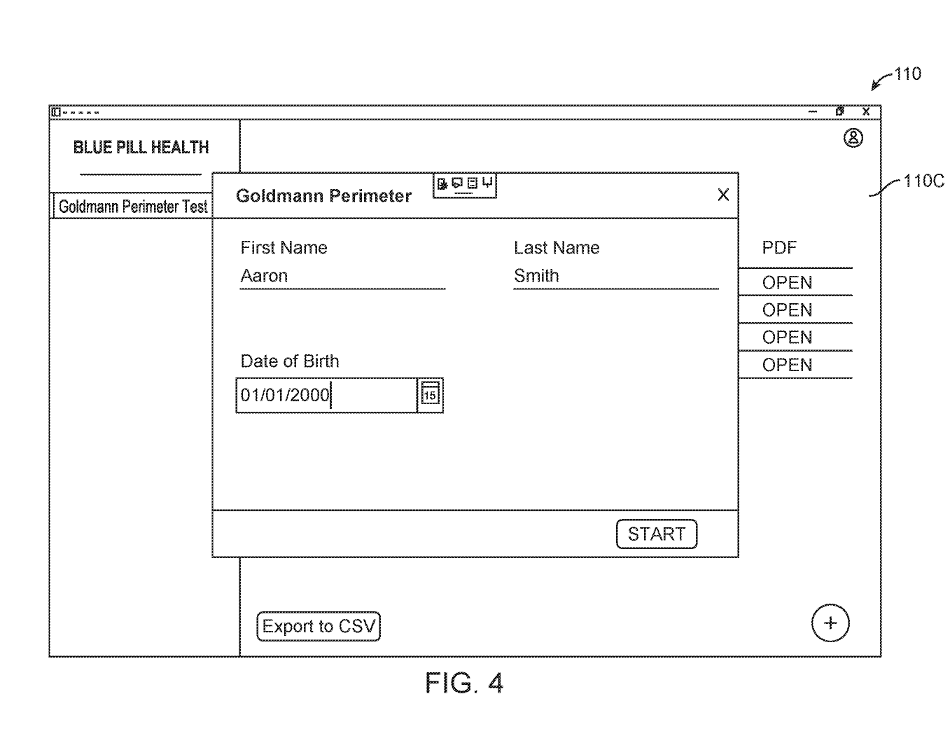

FIG. 4 illustrates an example user interface 110 according to implementations of the current subject matter. The user interface 110 illustrated in FIG. 3 shows an example progress display 110C. The progress display 110C can be displayed via the user interface 110 when the exam begins. The progress display 110C can show a current stage or the progress of the examination. In some implementations, the examination can be started, paused, restarted, or ended by selecting an option displayed on the progress display 110C.

As mentioned above, the virtual reality headset 114 can include the virtual reality engine 116. The virtual reality headset 114 can include any suitable headset 114, such as example headset configurations described herein. The virtual reality headset 114 can include at least one display screen 114A. The display screen 114A can present the test scene to the user of the virtual reality headset 114. In some implementations, the virtual reality headset 114 can include a flexible display screen 114A. The flexible display screen 114A can include light emitting diodes, and/or other light emitting material to present the virtual reality scene to the patient.

In some implementations, the virtual reality headset 114 can include one or more sensors 150 such as an eye-tracking device to determine the position of one or both eyes of the patient. The position of the user's eyes may determine, at least in part, the test scene presented to the patient on the display screen 114A. For example, when the patient's eyes move, the eye-tracking device may provide information to cause the display screen 114A to present at least a portion of the test scene. In some implementations, the one or more sensors 150 can include a switch or other sensor to provide a confirmation to the processor 120 for a selection made by the patient and/or user. In some implementations, the eye tracking device can determine whether one or both of the patient's eyes has lost fixation on the cross-hair (described herein) or other portion of the display scene presented to the patient. In such configurations, if the patient has lost fixation, the system 100 can stop the test and issue a command to alert the patient or notify the patient and/or user.

The virtual reality headset 114 can include one or more processors 120 and memory 122. The one or more processors 120 and/or memory 122 can generate the virtual reality scenes presented on the display screen 114A of the virtual reality headset 114 to be displayed to the patient. The one or more processors 120 and/or memory 122 can generate the virtual reality scenes based on information from the one or more sensors 150. The virtual reality scenes can be stored in memory 122. For example, the one or more processors 120 and/or memory 122 can include executable code that adjusts the scene on the display screen 114A of the virtual reality headset 114 according to information or other data received from the eye-tracking sensor 150, or other sensor, including an accelerometer, proximity sensor, and/or the like. For example, the one or more processors 120 and/or memory 122 can cause the virtual reality scene to change to a next step in the test.

In some implementations, the virtual reality headset 114 may include one or more transceivers 152. The transceivers 152 can include radio transceiver(s), optical transceiver(s) and/or wired transceiver(s). For example, virtual reality headset 114 can include a radio transceiver 152 to transmit and receive a radio signals to/from another transceiver at the display unit 108. The receiver in the transceiver 152 can receive an analog or digital representation of an audio signal generated by a microphone at the display unit, or other portion of the system 100. The transmit portion of the transceiver 152 may take an electrical signal generated by microphone 152 or digital representation of the generated signal and transmit the signal or digital representation to a receiver at the display unit 108. A receiver at the display unit 108 may regenerate the patient's voice at the display unit 108. The patient and/or the user may communicate via a wired and/or wireless connection between the transceivers 152. The transceiver 152 can use an antenna, and/or other wired or wireless connection means to transmit and receive signals corresponding to the audio and/or visual communications between the patient and/or user.

In some implementations, the display scene displayed at the display screen 114A of the virtual reality headset 114 may be duplicated at the display unit 108 and/or the display scene displayed at display unit 108 may be duplicated at the display screen 114A of the virtual reality headset 114. A transceiver 152 may transmit the display scene displayed at the display screen 114A of the virtual reality headset 114 to a receiver at the display unit 108 for viewing at the display unit 108. In some implementations, the bidirectional audio communications between the patient and/or the user, and the video send from virtual reality headset 114 to the display unit 108 (or vice versa) may use a single transceiver. In some example embodiments the transceiver may perform in accordance with a cellular communications standard (e.g., 2G, 3G, 4G, 5G, GSM, etc.), any of the WiFi family of standards, Bluetooth, WiMax, or any other wireless, wired, or optical communications standard.

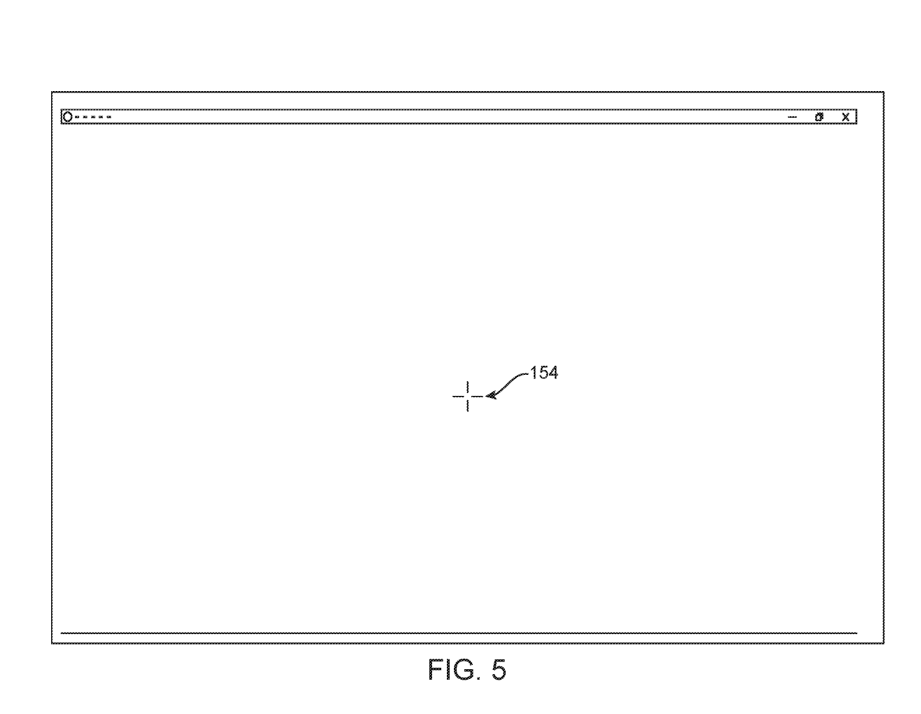

In some implementations, the virtual reality headset 114 can include the virtual reality engine 116. The virtual reality engine 116 can generate and/or display a test scene on the display screen of the virtual reality headset 114. The display scene can include a cross-hair 154 (e.g., a fixation point) at the center of the patient's vision and/or a stimuli, such as a color. An example of this configuration of the test scene is illustrated in FIG. 5. As shown in FIG. 5, the test scene is an example of a two-dimensional projection of the three-dimensional display scene the patient would see when the virtual reality headset 114 is worn by the patient. The display scene of FIG. 5 shows the fixation point at the center, with no stimuli being shown.

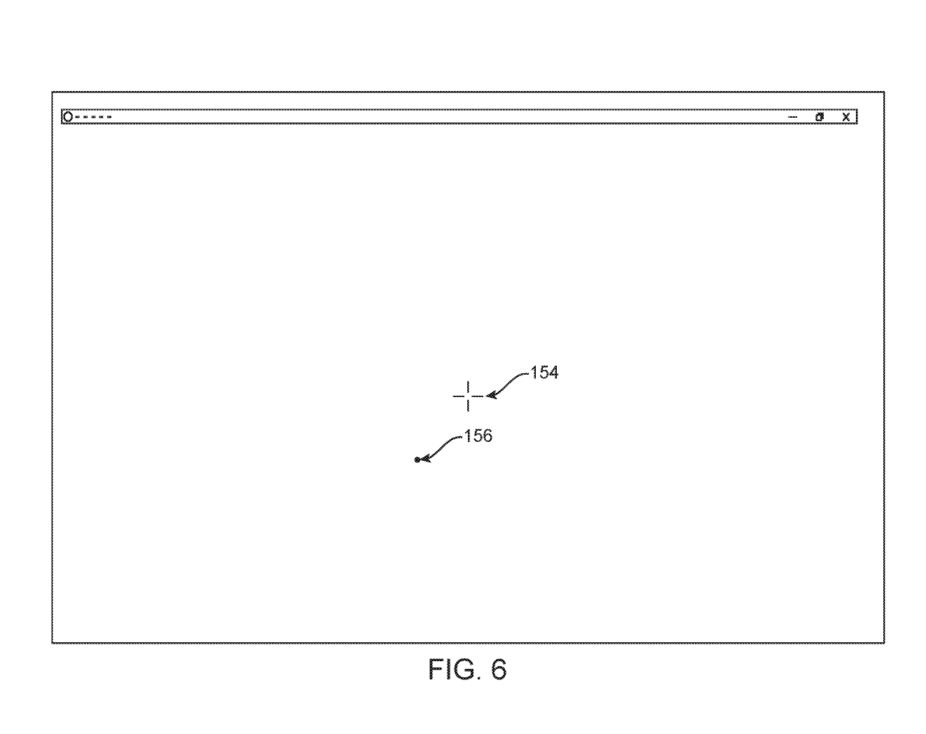

As described in more detail below, after the test begins (automatically, or manually), a stimulus 156, such as a color, can be displayed on the display scene. The stimulus can flash, translate from a periphery towards a center, and/or the like. The movement of the stimulus displayed to the patient can depend on whether a static or kinetic test is being administered to the patient. In some implementations, the stimulus flashes when the static test is being performed. In some implementations, the stimulus translates across the display screen 114A of the virtual reality headset 114 when a kinetic test is being performed. The stimulus is represented by a black dot as shown in FIG. 6. However, other configurations of the stimulus are contemplated. For example, rockets, birds, characters, or other objects can be used.

In use, the patient can actuate a control to indicate that the patient has seen the stimulus. For example, the patient can click at least a portion of the control to actuate the control. In some implementations, the control can include other touch-sensitive devices such as single or multi-point resistive or capacitive track pads. Such configurations can help to detect false positives. For example, the system 100 can compare the direction indicated on the touch-sensitive device to the actual location of the stimulus relative to the fixation point. A difference between the actual location of the stimulus relative to the fixation point, and the direction indicated by the patient would indicate a false positive.

The control can be connected to the virtual reality headset 114 and/or the display unit 108 through a wired and/or wireless connection. The system 100 can record data, such as the patient's responses, such as the user's actuations, and information about the user's responses according to methods described herein. The system 100 can determine whether the user's response is valid and/or invalid. The system 100 can determine whether another cross-hair, or stimulus should be displayed to the patient.

When the virtual reality headset 114 is worn by the patient, a lens of the virtual reality headset 114 can distort the image that is displayed to the patient. The lens can distort the image so that the image is displayed to the patient as the patient's actual field of view.

In some implementations, the system 100 can control which of the patient's eyes sees the stimulus that is displayed. For example, each of the patient's eyes can see a different display screen 114A of the virtual reality headset 114. In some implementations, if the right eye is being tested, the fixation point can be shown in both eyes, but the stimulus may only be shown to the patient's right eye. In some implementations, if the left eye is being tested, the fixation point can be shown in both eyes, but the stimulus may only be shown to the patient's left eye. In such configurations, the patient would desirably not need to close the eye that is not being tested. In some implementations, if the right and/or left eye is being tested, the fixation point can be shown in both eyes, and the stimulus may be shown to the patient's right and/or left eye.

In some implementations, the virtual reality environment displayed by the virtual reality headset 114 can be stabilized. In the stabilized virtual reality environment, the fixation point and/or the stimulus can remain in the same position on the display screen 114A of the virtual reality headset 114, regardless of the position of the patient's head. In some implementations, the test image displayed to the patient when the virtual reality headset 114 is worn by the patient can move with the patient's head, rather than remain stationary within the virtual reality environment. Such configurations can desirably account for movement of the patient's head when the test is being performed. Such configurations can desirably lead to more accurate measurements

In some implementations, when the test is completed, the administrative engine 112 can automatically disable the virtual reality engine 116. Before the virtual reality engine 116 is disabled, the virtual reality engine 116 can transmit the data measured by the virtual reality engine via the one or more sensors and/or the control actuated by the patient, to the administrative engine 112 and/or the database 106 to be stored. In some implementations, the virtual reality engine 116 can transmit the data to medical record database, such as a database that includes a patient's or a plurality of patient's medical records. In some configurations, the data can be linked to the patient's medical records so that the data is easily accessible by a physician or other user.

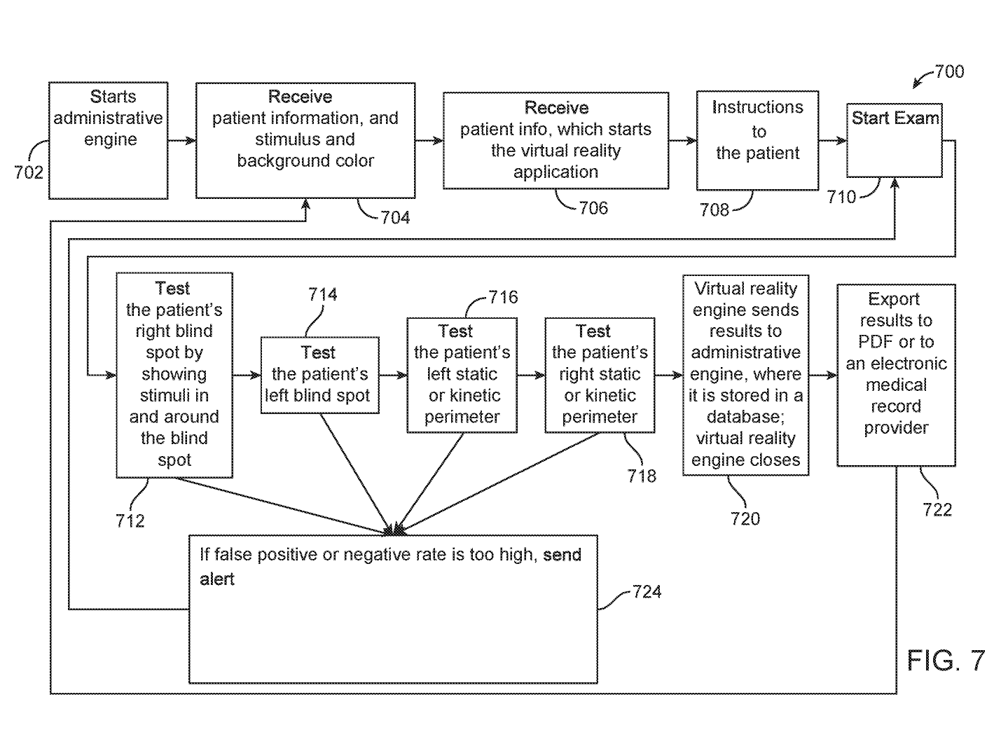

FIG. 7 illustrates an example method 700 for performing the visual field exam on the patient according to implementations of the current subject matter.

At 702, the user, such as a technician, can run the administrative engine 112 via the display unit 108. At 704, the user can begin the visual field exam by selecting an option displayed on the user interface 110 of the display unit 108. The user can enter in the patient's information, or search for, or otherwise retrieve the patient's information. The user can select using the user interface 110, the stimulus and/or the background color to be presented to the patient on the display screen 114A of the virtual reality headset 114. At 706, the user can submit the patient information, including any new patient information, and/or submit the color of the stimulus and/or the background of the display screen 114A of the virtual reality headset 114. In some implementations, the virtual reality engine 116 can automatically begin the examination once the patient information and/or the color information is submitted.

At 708, the user can instruct the patient verbally and/or electronically through the wired or wireless connection. The user can begin the exam at 710.

At 712, the virtual reality engine 116 can test at least one of the patient's eyes. For example, the virtual reality engine 116 can test at least the patient's right eye and/or right blind spot. The virtual reality engine 116 can cause the display screen 114A of the virtual reality headset 114 to present a testing scene to the patient. The first testing scene can show a stimulus in and/or around the blind spot. In some implementations, at 714, the virtual reality engine 116 can test at least the other of the patient's eyes, such as the left eye of the patient. The test for the other eye of the patient can be the same or similar to the test performed on the first eye of the patient.

After, or before the virtual reality engine 116 tests the patients blind spot in the patient's right and/or left eye, at 716 the virtual reality engine 116 can test the patient's right (or left) static and/or kinetic perimeter, by for example flashing, moving, or otherwise presenting the stimulus on the darker background in various locations. At 718, the virtual reality engine 116 can test the patient's other eye, such as the patient's left (or right eye). In some implementations, the system 100 can determine whether there is a false positive or a false negative in the patient's responses or data collected, after the virtual reality engine 116 tests the left and/or right eye for the blind spot and/or the static or kinetic perimeter. At 724, the system 100 can determine whether the false positive and/or negative rate is too high. The virtual reality headset 114 and/or the display unit 108 can issue a notification to alert the user. The user can administer a warning and/or restart the exam. The false positive and/or negative rate can be determined and/or otherwise analyzed using the eye tracking device. In some implementations, the false positive and/or negative rate can be determined by showing stimuli in known visible and/or blind spots.

In some implementations, if the false positive and/or negative rate is too high, the virtual reality engine 116 and/or the administrative engine 112 can alert the user, who can administer a warning and/or restart the exam. In some implementations, at 720, the virtual reality engine 116 can transmit the results of the exam to the administrative engine 112 through means described herein. The results can be stored in the database 106. In some implementations, as described above, the virtual reality engine 116 can disable the virtual reality headset automatically after the results are transmitted to the display unit 108 and/or the results can be manually transmitted to the display unit 108. In some implementations, at 722, the user can export the results to a report format, such as in a graph, chart, and/or the like. In some implementations, the results can be exported and/or otherwise transmitted to an electronic medical record provider.

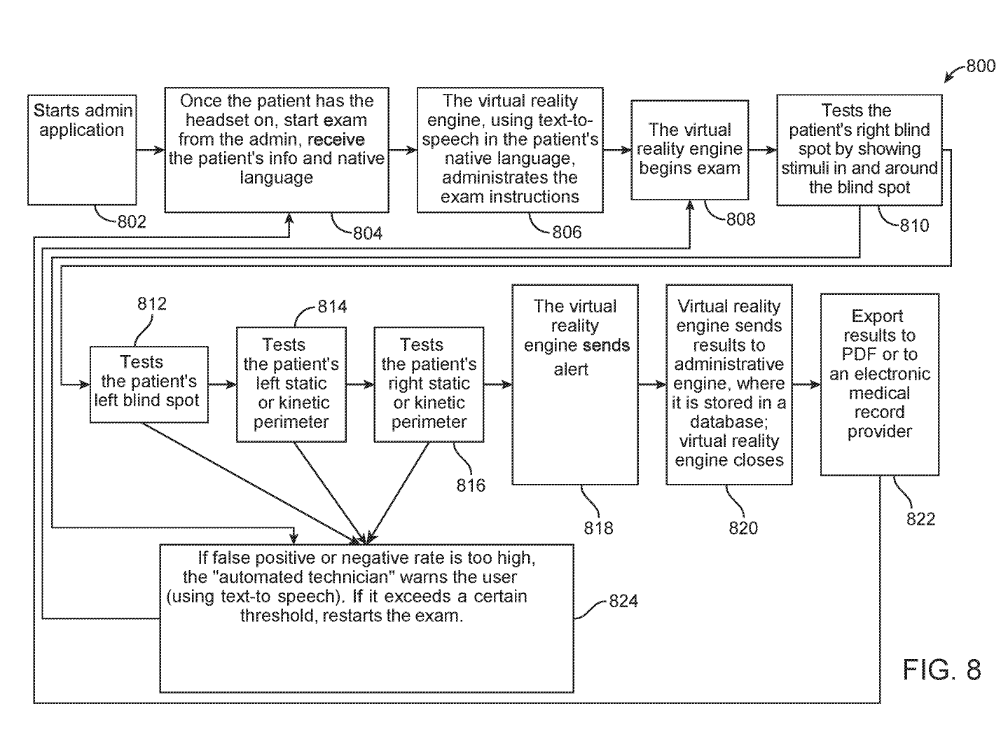

FIG. 8 illustrates an example method 800 for performing the visual field exam on the patient according to implementations of the current subject matter. In some implementations, the virtual reality engine 116 can provide instructions to the patient before and/or after the exam begins and/or to correct fixation loss if the patient's focus drifts during the exam, among other functions. As explained in more detail below, if the user lost focus, the virtual reality engine 116 can pause or stop the exam. In some implementations, the virtual reality engine 116 can, in the native language of the patient for example, direct the patient to focus on the fixation point. Such configurations can desirably provide a faster exam procedure and/or lead to more accurate test results. Such configurations can desirably eliminate the need for a specially trained technician, physician or other professional. Such configurations can desirably allow the patient to use the virtual reality headset 114 to take the visual field exam without the need to travel to a medical facility, saving a significant amount of time and expense.

At 802, the user, such as a technician, can run the administrative engine 112 via the display unit 108. At 804, the user can begin the visual field exam by selecting an option displayed on the user interface 110 of the display unit 108. The user can enter in the patient's information, or search for, or otherwise retrieve the patient's information. The user can select using the user interface 110, the stimulus and/or the background color to be presented to the patient on the display screen 114A of the virtual reality headset 114. In some implementations, the user can select and/or otherwise input the patient's language.

At 806, the user can submit the patient information, including any new patient information, and/or submit the color of the stimulus and/or the background of the display screen 114A of the virtual reality headset 114 or the patient's language. The stimulus and/or background color choice can be stored in a database described herein to be accessed by the patient and/or user, and/or be stored with the exam results when the exam is completed. The stimulus and/or background color choice can be sent to the virtual reality engine 1116 to set the color scheme on the virtual reality headset display screen 114A.

In some implementations, the virtual reality engine 116 can automatically begin the examination once the patient information and/or the color information is submitted, and/or when the patient places the virtual reality headset 114 on their head in the proper position. In some implementations, the virtual reality engine 116 can administer a set of exam instructions by playing a recording in the selected language, by converting text instructions to audible instructions, and/or the like.

At 808, the virtual reality engine 116 can begin the exam.

At 810, the virtual reality engine 116 can test at least one of the patient's eyes. For example, the virtual reality engine 116 can test at least the patient's right eye and/or right blind spot. The virtual reality engine 116 can cause the display screen 114A of the virtual reality headset 114 to present a testing scene to the patient. The first testing scene can show a stimulus in and/or around the blind spot. In some implementations, at 812, the virtual reality engine 116 can test at least the other of the patient's eyes, such as the left eye of the patient. The test for the other eye of the patient can be the same or similar to the test performed on the first eye of the patient.

After, or before the virtual reality engine 116 tests the patients blind spot in the patient's right and/or left eye, at 814 the virtual reality engine 116 can test the patient's right (or left) static and/or kinetic perimeter, by for example flashing, moving, or otherwise presenting the stimulus on the darker background in various locations. At 816, the virtual reality engine 116 can test the patient's other eye, such as the patient's left (or right eye). In some implementations, the system 100 can determine whether there is a false positive or a false negative in the patient's responses or data collected, after the virtual reality engine 116 tests the left and/or right eye for the blind spot and/or the static or kinetic perimeter. At 824, the system 100 can determine whether the false positive and/or negative rate is too high. The virtual reality headset 114 and/or the display unit 108 can issue a notification to alert or otherwise warn the user and/or patient. The virtual reality engine 116 can compare the rate of false positives and/or negatives to a threshold value. If the rate of the false positives and/or negatives exceeds the threshold, the virtual reality engine 116 can terminate and/or restart the exam. The false positive and/or negative rate can be determined and/or otherwise analyzed using the eye tracking device. In some implementations, the false positive and/or negative rate can be determined by showing stimuli in known visible and/or blind spots.

In some implementations, at 818, the virtual reality engine 116 can alert the user and/or the patient to remove the virtual reality headset 114. In some implementations, at 820, the virtual reality engine 116 can transmit the results of the exam and/or the stimulus and/or background color to the administrative engine 112 through means described herein. The results can be stored in the database 106. In some implementations, as described above, the virtual reality engine 116 can disable the virtual reality headset automatically after the results are transmitted to the display unit 108 and/or the results can be manually transmitted to the display unit 108. In some implementations, at 822, the user and/or the virtual reality engine 116 can (e.g., automatically) export the results to a report format, such as in a graph, chart, and/or the like. In some implementations, the results can be exported and/or otherwise transmitted to an electronic medical record provider.

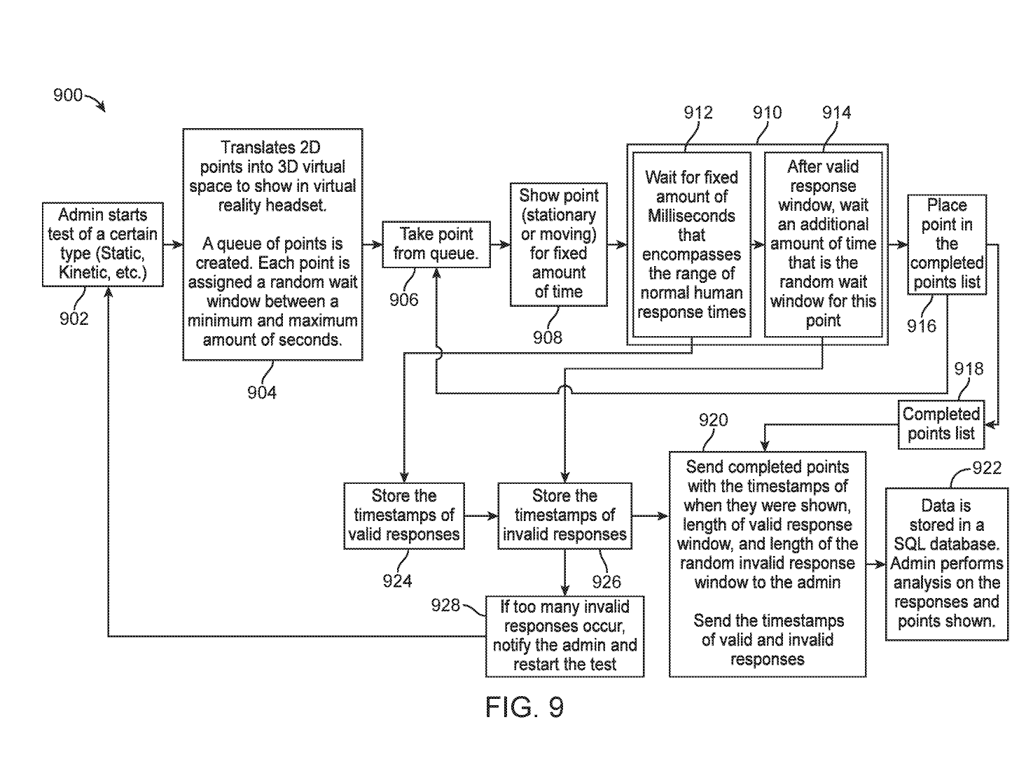

FIG. 9 illustrates an example method 900 of capturing measurements or other readings taken during the visual field exam, according to implementations of the current subject matter. At 902, the administrative engine 112 begins the test. At 904, the virtual reality engine 116 can translate two-dimensional points (such as the stimulus) to three-dimensional points to display to the patient via the display screens 114A of the virtual reality headset 114. The points can be stored within a queue. The positions of the points can be randomly assigned and/or can be predetermined. In some implementations, the time at which the points are displayed to the patient are randomly assigned and/or preset. In some implementations, each point is assigned a wait window between a minimum and/or maximum amount of time for the points to be presented to the patient.

At 906, the virtual reality engine 116 can retrieve the next point from the queue of points. At 908, the virtual reality engine 116 can present the point. The point can be presented to the patient as a stationary and/or moving point depending on the type of test, as described herein. The point can be presented to the patient for a fixed amount of time, such as the amount of time assigned to the point in the wait window.

At 910, the visual field exam can enter a waiting period. In the waiting period, at 912, the virtual reality engine 116 can present the point to the patient for valid response time window (e.g., in milliseconds) that encompasses a range at which an average human responds to seeing the stimulus presented on the display screen 114A of the virtual reality headset 114. The average time can be approximately 1-2 milliseconds, 3-4 milliseconds, 4-5 milliseconds, and/or more.

At 924, the virtual reality engine 116 can measure and/or store timestamps corresponding to the time at which the control received a valid response from the patient. The timestamps can be stored in a database within the virtual reality headset 114 and/or in a remote database, such as a database stored in the display unit 108 and/or another remote database. At 924, the virtual reality engine 116 can measure and/or store timestamps corresponding to the time at which the control received an invalid response from the patient. The timestamps can be stored in a database within the virtual reality headset 114 and/or in a remote database, such as a database stored in the display unit 108 and/or another remote database. If too many invalid responses are received, the virtual reality engine 116 can notify the patient and/or the user. The virtual reality engine can restart the exam at 928 upon receiving a greater number of invalid responses than a threshold number of invalid responses.

At 914, after the valid response time window, the virtual reality engine 116 an wait an additional amount of time that is equal to the preassigned time window that was assigned to the point at 908. In some implementations, the method 900 can repeat blocks 926 and/or 928.

At 916, after the waiting period, the point can be stored in a completed points list and/or queue. In some implementations, the completed points list can be completed at 918. At 920, the completed points and/or other measured information can be transmitted to the administrative engine 114 of the display unit 108. The measured information can include the timestamp corresponding to the completed point, the timestamp of the valid and/or invalid response, the timestamp of the time the point was presented to the patient, the length of the valid response window, the length of the random invalid response window, and/or the like.

At 922, the measured information can be stored in a database located in the virtual reality headset 114, the display unit 108 and/or another database. The database can be an SQL database. The administrative engine 114 can determine the patient's visual field based on the measured information at 922.

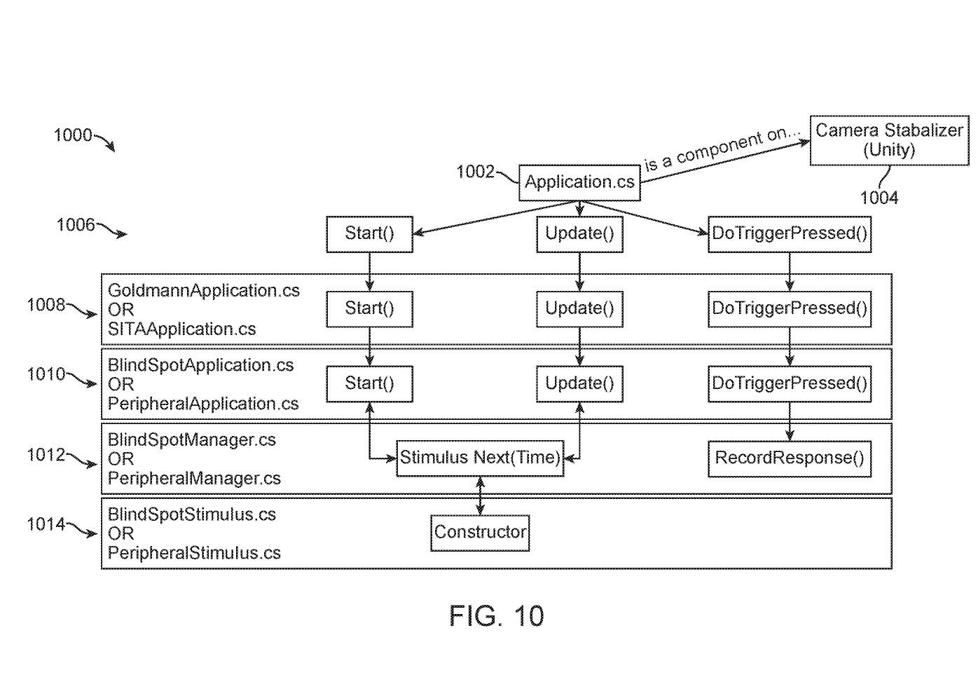

FIG. 10 illustrates an example virtual reality visual field exam architecture 1000. The architecture 1000 can include a plurality of data layers, such as a first layer 1006, a second layer 1008, a third layer 1010, a fourth layer 1012, and/or a fifth layer 1014, among others. The first layer 1006 can switch between test section type. For example, the first layer 1006 can be told the type of test to run by the administrative engine 114 and can be the exam on the virtual reality headset 114 via the virtual reality headset engine 116. The second layer 1008 can manage the flow of the selected test section or test type. For example, the second layer 1008 can have a queue of test sections to run (e.g., right and/or left blind spot and/or right and/or left peripheral). The second layer can run the test sections sequentially in some implementations.

The third layer 1010 can manage an individual test section. For example, the third layer can present a first blind spot point, then a second blind spot point, and/or other blind spot or peripheral points. The third layer 1010 can have a queue of managers to run. The third layer 1010 can call Next( ) on the current manager to retrieve a point to present to the patient. When the point has been presented to the patient for the desired amount of time, the third layer 1010 calls Next( ) again to retrieve the next point in the queue.

The fourth layer 1012 can track the upcoming and/or completed points, and/or patient responses. The fourth layer 1012 can have a queue of points to present to the patient. Each time Next( ) is called, the fourth layer 1012 saves the previous point and returns the next point. If RecordResponse( ) is called, the fourth layer can record the patient's response in a list.

The fifth layer 1014 can include the stimulus. The fifth layer can represent an individual point. Each point has an amount of time the pint should be presented (e.g., for static tests), a starting and ending point (e.g., for kinetic tests), and windows of time within which to expect valid or invalid responses. A stimulus can be different for each exam and/or test section. The stimulus can include a point where the stimulus is shown and/or a point the stimulus should move to, along with corresponding information such as how long the stimulus should be presented, and/or how long to wait after the stimulus is presented.

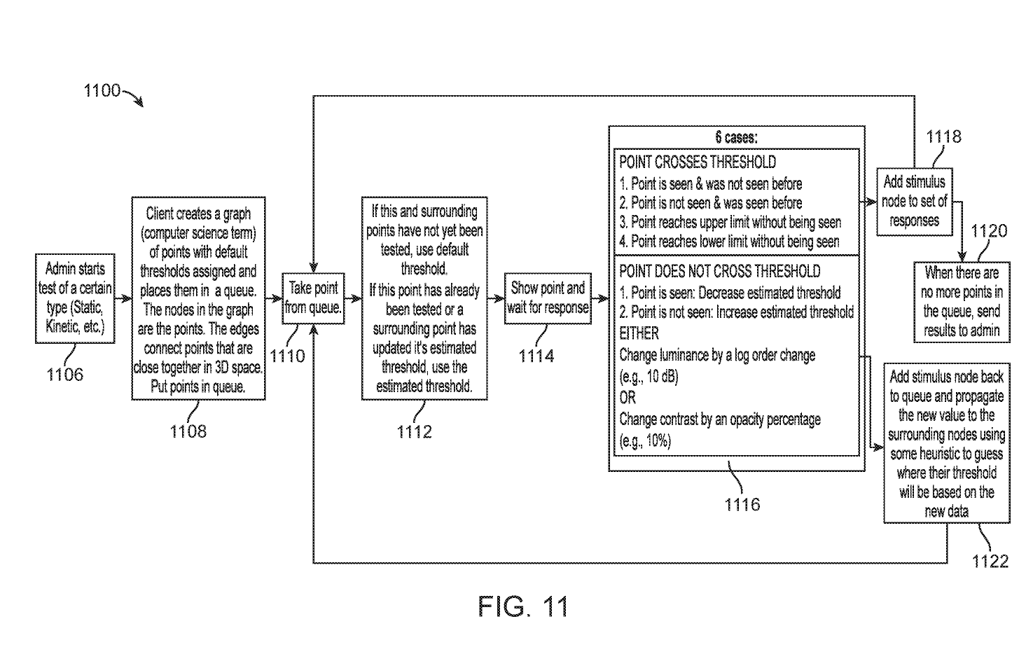

FIG. 11 illustrates an example method 1100 of using luminance to determine a patient's threshold. The patient's threshold can be the amount of light for the patient to see. Typically, luminance is modified on a log scale, so every increase of 10 dB is an increase of many orders of magnitude. In some implementations, luminance can be used as a measure for eye sensitivity and can be used to indicate one or more of the ocular disorders discussed herein. For example, eye sensitivity can be tested by showing a black stimulus on a white background, then a gray stimulus on a white background, among other configurations. In some implementations, in using luminance the brightness of a pixel or group of pixels on the display screen 114A of the virtual reality headset 114 can be varied. In some implementations, contrast can be used to test for certain ocular disorders discussed herein by varying the color contrast between a group of pixels and the background.

As shown in FIG. 11, at 1106, the administrative engine 112 can begin the test of a certain type (e.g., static or kinetic). At 1108, the virtual reality engine can create a graph of points with default thresholds assigned. The virtual reality engine 116 can place the points in a queue. The graph can include a plurality of nodes. The nodes of the graph can define the points. In some implementations, edges of the graph connect points that are close together in a three-dimensional space. The virtual reality engine 116 can place the points into the queue. At 1110, the virtual reality engine 116 can retrieve a point from the queue.

At 1112, if the retrieved point and/or surrounding points have not yet been tested, the default threshold can be used. If the retrieved point has already been tested and/or a surrounding point has updated the estimated threshold, the estimated threshold should be used.

At 1114, the virtual reality engine 1116 can present the retrieved point to the patient. At 1116, the virtual reality engine 1116 can determine whether the point crosses the threshold. For example, the point can cross the threshold when the point is seen and was not seen before, the point is not seen and was seen before, the point reaches an upper limit of the graph without being seen, and/or the point reaches the lower limit of the graph without being seen. In some implementations, the point may not cross the threshold. For example, if the point is seen, the virtual reality engine 1116 can decrease the estimated threshold. If the point is not seen, the virtual reality engine 1116 can increase the estimated threshold. The luminance can be changed by a log order change (e.g., 10 dB) and/or the contrast can be changed by an opacity percentage (e.g., 10%).

When the point crosses the threshold, the stimulus node can be added to a set of responses at 1118. After the stimulus node is added to the set of responses, another point can be retrieved from the queue at 1110. When there are no more points in the queue, the results can be sent to the administrative engine 1114 at 1120.

At 1122, if the point does not cross the threshold, the stimulus node can be added to the queue and a new value can be propagated to the surrounding nodes to estimate where the point's threshold will be based on the measured information. After the stimulus node is added back to the queue, another point can be taken from the queue at 1110.

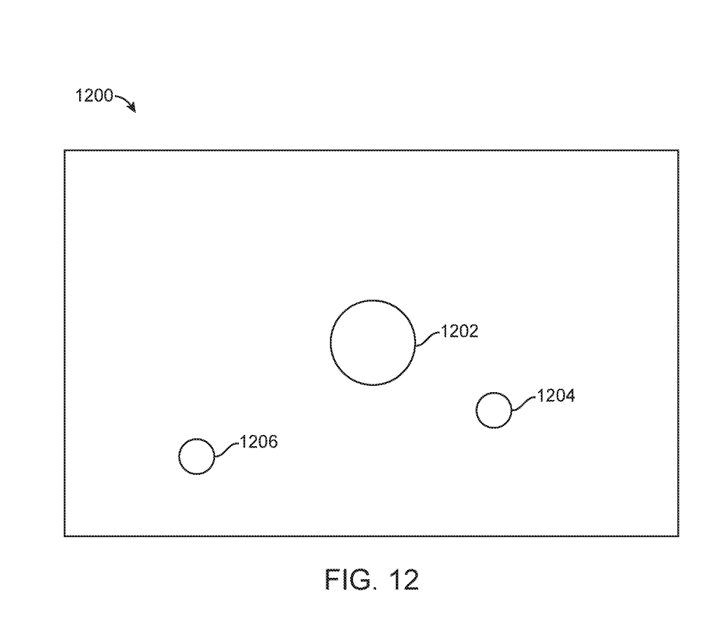

FIG. 12 illustrates an example visual field schematic 1200 that depicts possible features in the right eye of a patient's vision. The visual field concept 1200 illustrates a macular area 1202, a blind spot 1204, and a scotoma 1206. For example, the macular area 202 may be seen by a center of a patient's retina, and may be the most sensitive part of the patient's field of vision. In the patient's eye, the macula generally has a diameter of about 1.5 mm. The macular area 1202 in the patient's vision may have a diameter of about 5 degrees. The blind spot 1204 may be located spaced away from the macular area 1202, about 15 degrees temporally and 1.5 degrees below the horizontal away from the macular area 1202. The blind spot 1204 may be about 7.5 degrees high and 5.5 degrees wide. In the example shown in FIG. 12, the blind spot 1204 is on the right hand side. In other implementations depicting the visual field in the left eye of the patient's vision, the blind spot 1204 may be on the left hand-side of the depiction. The scotoma 1206 is shown nasally in this example, which as noted above, indicates an unnatural blind spot that may appear anywhere in a patient's vision as a result of an ocular disease.

In some implementations, the system 100 may be used for relative stimuli presentation. As noted above, the systems described herein relate to testing a patient's visual field, such as by presenting various stimuli (e.g., represented by a stimulus vector) and measuring and/or otherwise analyzing the patient's gaze (e.g., during static and/or kinetic thresholding). As noted above, the stimuli displayed by the virtual reality engine 116 via the display of the virtual reality headset 114 may be shown relative to a fixation point of the patient's gaze, which may be defined by a gaze vector. In some implementations, both the vertical and horizontal angles between the gaze vector and the stimulus vector may be the same, regardless of where the patient's gaze is located (e.g., the location where the patient fixates). If the patient's gaze changes during the examination, the virtual reality engine 116 may display stimuli relative to the patient's gaze so that the same test may be provided regardless of where the patient looks on the display. This may allow for the test implemented via the virtual reality headset 114 to be used consistently across various patients.

Generally, in static and kinetic perimetry, stimuli are shown in fixed positions, as noted above, in absolute space, regardless of where the patient looks. In such testing methods, if the patient looks away from the fixation point, the test may generate fixation loss errors, thereby resulting in invalid visual field results.

The visual field analyzer systems described herein may allow for and/or account for the patient fixating in various locations (e.g., not at a single fixation point), and may instead allow for the patient's gaze to drift to a location that is comfortable for the patient. Such systems allow for greater accuracy and consistency in applying the visual field testing methods described herein, improving the results of the testing. For example, the virtual reality engine 116 may display the stimuli on the display screen 114A of the virtual reality headset 114 in the same relative positions, such as, by calculating relative values based on the direction of the patient's eye(s) via one or more eye tracking mechanisms described herein.

In some implementations, the visual field analyzer system 100 described herein can perform relative stimuli presentation. For example, as noted above, the virtual reality engine 116 can display stimuli relative to the patient's gaze. The virtual reality headset 114 may include the eye tracking mechanisms to track the patient's gaze.

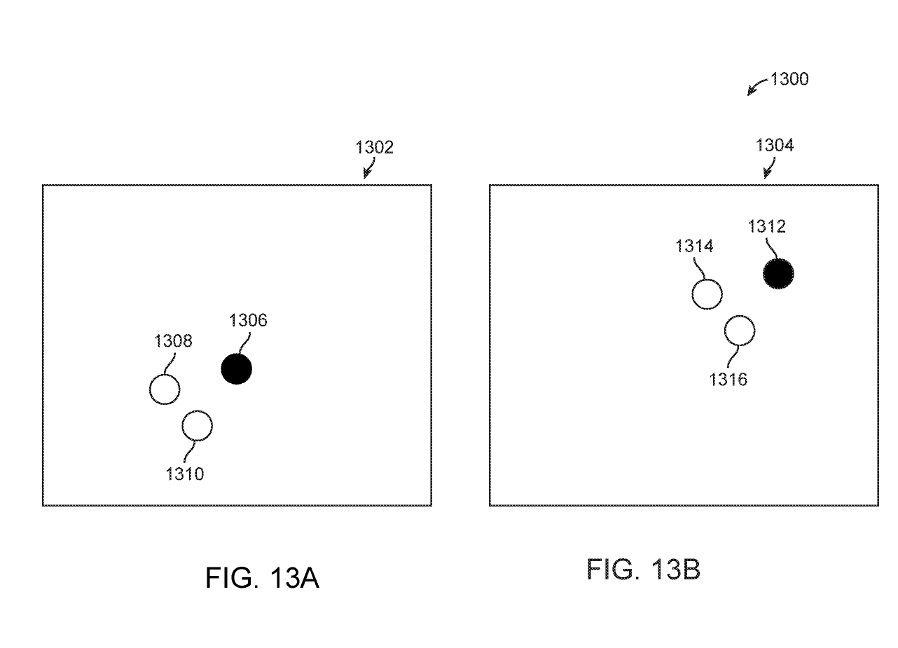

FIGS. 13A and 13B illustrate example test scenes presented to the patient via the display screen 114A of the virtual reality headset 114, during relative gaze presentation, consistent with implementations of the current subject matter. For example, FIG. 13A shows a gaze diagram 1302 illustrating relative gaze tracking. In the gaze diagram 1302, the patient's gaze is primary focal point 1306. The virtual reality engine 116 may display stimulus 1308 and stimulus 1310 relative to the focal point 1306. FIG. 13B shows another gaze diagram 1304, illustrating relative gaze tracking. In the gaze diagram 1304, the patient's gaze is shown as alternative focal point 1312. The virtual reality engine 116 may display stimulus 1314 and stimulus 1316 relative to the focal point 1312. In both gaze diagrams 1302 and 1304, the two stimuli pairs (e.g., stimulus 1308, 1310 and stimulus 1314, 1316) shown in each diagram stimulate the same locations on the patient's retina.

In some implementations, the system 100 may be used for responsive gaze tracking, during static and/or kinetic perimetry, and the like. Generally, using the system described herein, the patient may look at a stationary fixation point in their field of view on the display screen 114A of the virtual reality headset 114. Using eye tracking, the system 100 monitors the patient's gaze. If the patient's gaze travels beyond a pre-defined boundary, then the virtual reality engine 116 can pause the in-progress test and/or notify the technician/user, and redirect the patient back to the fixation point, through text to speech, visual directions, on-screen text, or another type of indicator.

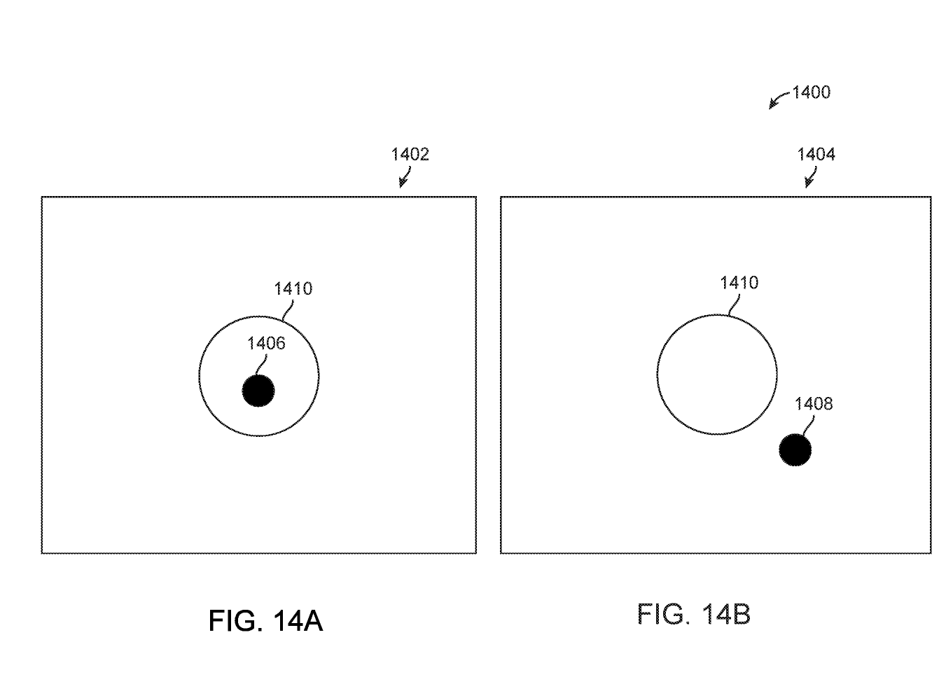

FIGS. 14A and 14B illustrate example test scenes presented to the patient via the display screen 114A of the virtual reality headset 114, during responsive gaze tracking, consistent with implementations of the current subject matter. For example, FIG. 14A shows a gaze diagram 1402 illustrating responsive gaze tracking. In the gaze diagram 1402 (e.g., illustrating a fixated gaze), the patient's gaze 1406 is shown as within a predefined acceptable radius 1410, which allows the virtual reality engine 116 to perform the testing. FIG. 14B shows another gaze diagram 1404 (e.g., illustrating an un-fixated gaze) illustrating responsive gaze tracking. In the gaze diagram 1404, the patient's gaze 1408 is shown as being outside the predefined acceptable radius 1410. The virtual reality engine 116 may pause, and/or stop the testing when the patient's gaze 1408 is measured by the system to be outside of the predefined acceptable radius 1410. When the testing is paused, for example, the virtual reality engine 116 may not present any stimuli to the patient via the display screen 114A of the virtual reality headset 114. The virtual reality engine 116 may indicate to the patient and/or the user that the patient's gaze 1408 is outside of the predefined acceptable radius 1410 to redirect the patient's gaze to an area within the predefined acceptable radius 1410.

In some implementations, the system 100 may map a patient's scotoma, for example, after or as part of a visual field test (e.g., the visual field tests described herein including static and/or kinetic thresholding). The system 100, such as via scotoma mapping, may be used to diagnose and/or monitor one or more ocular diseases described herein, such as macular degeneration and glaucoma, and/or to measure a size of the patient's blind spot. In some implementations, scotoma mapping may be performed by the system 100 automatically.

In some implementations, during automated scotoma mapping, one or more points within the patient's vision are supplied to the display screen 114A of the virtual reality headset 114 via the virtual reality machine 116. The one or more points may be supplied manually, such as when the display unit 108 receives a selection of points on a representation of the visual field via the administrative engine 112, and/or automatically, such as by a prior automated examination where thresholds deviated from the expected location.

During scotoma mapping, the patient may respond when they see a light or other stimulus. In some implementations, the patient may verbally respond, use a user interface (e.g., a clicker) in communication with the system 100, with their gaze via the display screen 114A of the virtual reality headset 114, and the like.

The virtual reality engine 116 may select a starting point or stimuli, such as one of the suspicious points that were provided to it. The point may be inside a “valley” or “depression” in the visual field. A “valley” in the visual field may include an area of statistically significant deviation in the corresponding patient's visual field results when compared to the visual field results from a group of healthy patients.

Using the starting point (e.g., which indicates a minimum brightness that the patient can see at that point in their vision), the virtual reality engine 116 selects one or more points of varying distances from the starting point and determines the minimum brightness of each of the one or more points. The virtual reality engine 116 may repeat this process using stimuli at increasing or decreasing distances from the original point until the virtual reality engine 116 determines the extent of the “valley” or “depression” in the patient's visual field. The edges of such a “valley” are where the patient's sensitivity to the stimuli returns to that of a standard or patient baseline, or otherwise reaches the outer boundaries of the test. Some points may not need to be retested if the points were already tested as part of a prior exam, such as during a kinetic and/or or static thresholding exam (as described herein).

In some implementations, at a single location, multiple stimuli may be shown. Each of the stimuli may vary in their color and/or intensity, as determined by the virtual reality engine 116. The virtual reality engine 116 may locate the minimum brightness the patient will respond to at that point.

The virtual reality engine 116 may present the test results on a graph or other visualization tool via the user interface, for example. The visualization tool may indicate the exact depth and edges of where the patient's vision deviates from the baseline. Thus, the system 100 can beneficially compare the progression of a patient's vision loss over time and indicate changes in the size, shape, and/or depth of the tested areas of vision loss.

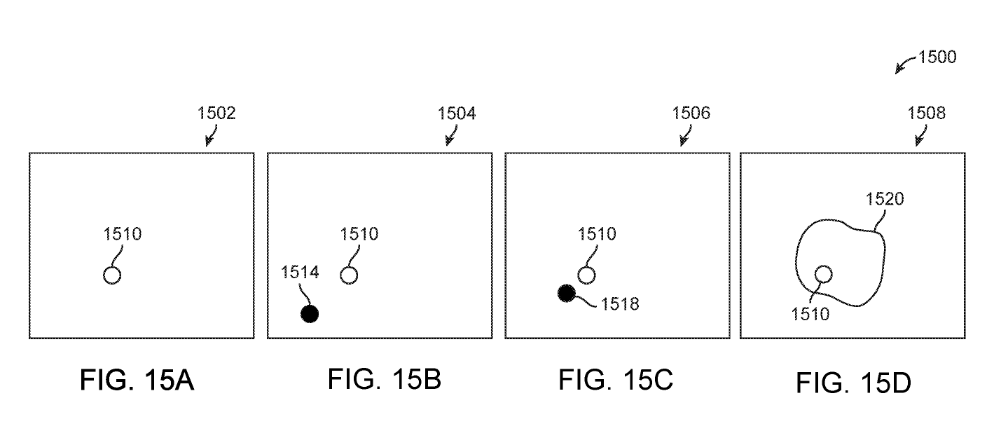

FIGS. 15A-15D illustrate various test scenes that may be displayed by the virtual reality engine 116 via the display screen 114A of the virtual reality headset 114 during automated scotoma mapping, consistent with implementations of the current subject matter. FIG. 15A illustrates an example starting test scene 1502 showing a possible stimulus location 1510. In some implementations, the possible stimulus location 1510 may have been identified during a prior exam, and may be stored in the database. As shown in a second test scene 1504 in FIG. 15B, the virtual reality engine 116 may determine a sensitivity for a nearby location 1514 by displaying a stimuli at the location 1514 via the display screen 114A of the virtual reality headset 114. As shown in a third test scene 1506 in FIG. 15C, the virtual reality engine 116 may then test another location 1518, which in this example, may be closer to the original possible stimulus location 1510. In some implementations, the virtual reality engine 115 may test one, two, three, four, five, or more locations by displaying stimuli at each of the tested locations on the display screen 114A of the virtual reality headset 114. After testing the stimuli at the one or more locations relative to the possible stimulus location 1510, the virtual reality engine 116 may identify a possible region 1520 (as shown in test scene 1508 in FIG. 15D). The virtual reality engine 116 may communicate with the administrative engine 112 at the user interface to report the possible region 1520 to the user. Points/locations within possible region 1520 may be tested individually (e.g., by the virtual reality engine 116) to determine the depth of the possible area indicating a scotoma.

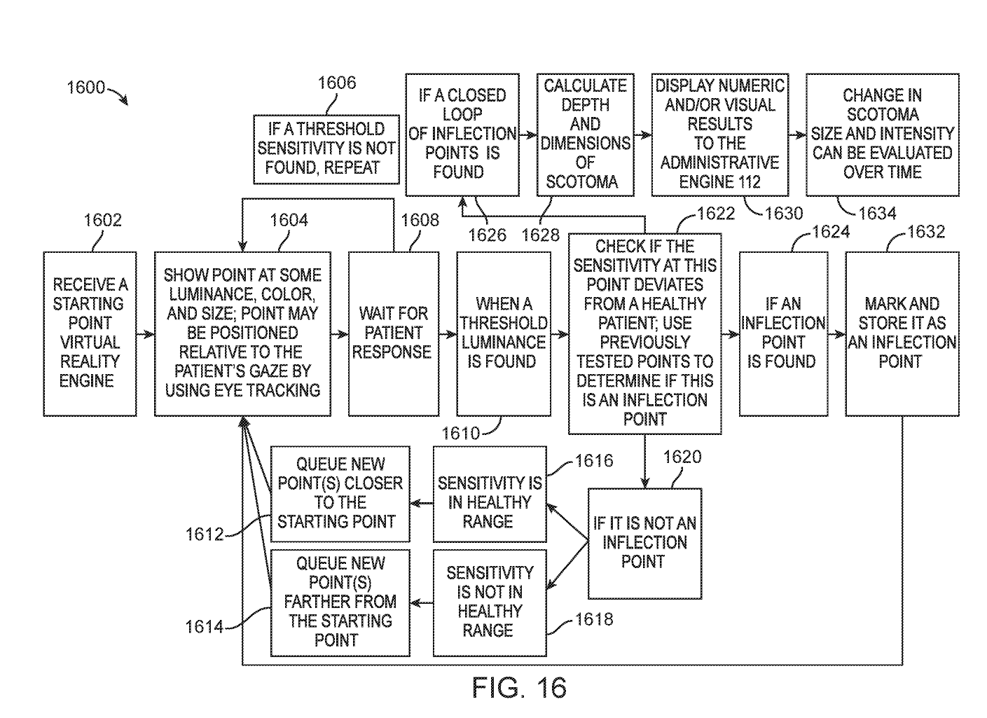

FIG. 16 illustrates an example method 1600 for performing automated scotoma mapping on the patient according to some implementations of the current subject matter.

At 1602, the virtual reality engine 116 may receive a possible stimulus location (e.g., location 510) to display via the display screen 114A of the virtual reality headset 114. At 1604, the virtual reality engine 116 may display the stimulus at the location at a selected luminance, color, and/or size. The stimulus may be positioned relative to the patient's gaze, by for example using eye tracking, as described herein.

At 1608, the virtual reality engine 116 may wait to receive a patient's response. At 1606, if no patient response is found (e.g., if a threshold sensitivity to the stimulus is not found), the virtual reality engine 116 may again display the stimulus at the location.

At 1610, the virtual reality engine 116 may determine that a patient response is found (e.g., the threshold luminance is determined). If the patient response is found, the virtual reality engine 116 may check whether the sensitivity at the location deviates from a healthy and/or baseline. The virtual reality engine 116 may compare the location and/or luminance to previously tested locations to determine whether the location is an inflection point, at 1622.

At 1626, the virtual reality engine 116 may determine that a closed loop of inflection points is found. If a closed loop of inflection points is found, at 1628, the virtual reality engine 116 may calculate a depth and/or dimension of the patient's scotoma. At 1630, the virtual reality engine 116 may communicate with the administrative engine 112 to display numeric and/or other visual representations of the results at the user interface. At 1634, the system 100 may use the collected information to evaluate the change in the patient's scotoma (e.g., size and/or intensity) over time.

At 1624, the virtual reality engine 116 may determine that an inflection point is found. If the inflection point is found, at 1632, the virtual reality engine 116 may store the location as the inflection point, for example, in the database. In some implementations, once an inflection point is found, the virtual reality engine 116 may display another point via the display at 1606.

In some implementations, at 1620, the virtual reality engine 116 may determine that the location is not an inflection point. If the virtual reality engine 116 determines that the possible stimulus location is not an inflection point, the virtual reality engine 116 may determine whether the sensitivity of the stimulus shown at the possible stimulus location is within a healthy range, or above a certain threshold. For example, at 1616, the virtual reality engine 116 may determine that the sensitivity of the stimulus is within the healthy range. If the virtual reality engine 116 determines that the sensitivity is within the healthy range, at 1612, the virtual reality engine 116 may queue one or more additional points/locations closer to the possible stimulus location (or previous possible stimulus location). The virtual reality engine 116 may then display the additional point at 1604. In some implementations, at 1618, the virtual reality engine 116 may determine that the sensitivity of the stimulus is not within the healthy range. If the virtual reality engine 116 determines that the sensitivity is not within the healthy range, at 1614, the virtual reality engine 116 may queue one or more additional points/locations farther from the possible stimulus location (or previous possible stimulus location). The virtual reality engine 116 may then display the additional point at 1604.

In some implementations, the system 100 may be used for eccentric preferred retinal locus (PRL) training, consistent with implementations of the current subject matter. For example, the system 100 may be used to help patients choose an eccentric PRL. In some implementations, the system 100 may be used to train patients to use the new PRL in their daily lives.

Generally, the PRL of patients with normal vision is generally at the very center of their vision (e.g., at the center of the macula). For patients with age-related macular degeneration (ARM) or maculopathy, the center of their vision may be deteriorated. Those patients may choose a new, or eccentric, preferred retinal locus in order to read and/or perform daily tasks.

As noted above, the system 100 may be used to help a patient identify an eccentric PRL that would be most useful to the patient. In some instances, because the patient's vision may not deteriorate in a perfect circle, an eccentric fixation point may be closer to the center of the patient's vision, while another eccentric fixation point may be farther away from the center of the patient's vision.

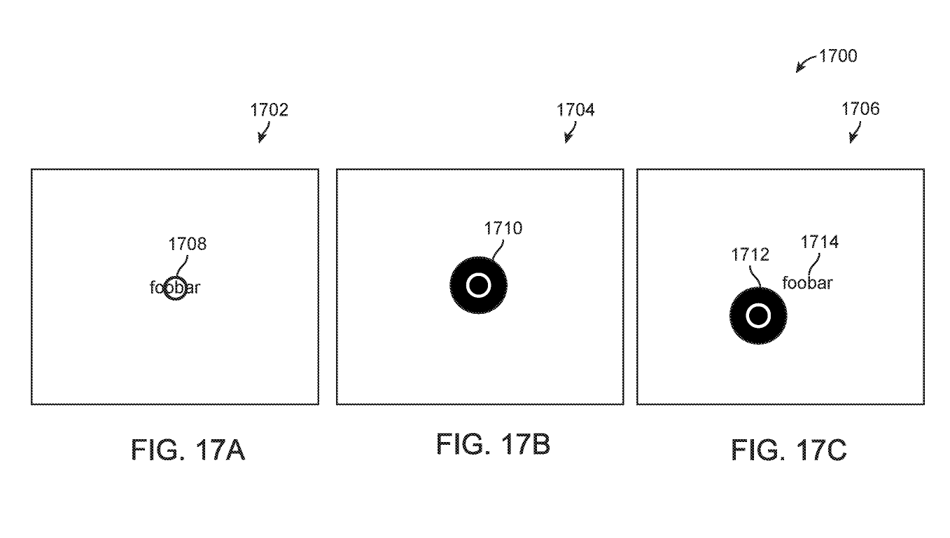

FIGS. 17A-17C illustrate example screens 1700 shown to the patient via the display screen 114A of the virtual reality headset, consistent with implementations of the current subject matter. As shown in FIGS. 17A-17C, the screens 1700 include a background showing the eccentric preferred retinal locus. FIG. 17A illustrates an example of a healthy patient's visual field 1702. In the healthy patient's visual field 1702, a patient's fixation point 1708 may be positioned directly on top of a piece of text (e.g., the text, “foobar”, is shown). FIG. 17B illustrates an example of an unhealthy patient's visual field 1704 (e.g., a patient having a disease, such as macular degeneration, maculopathy, and the like). In the unhealthy patient's visual field 1704, a natural fixation point 1710 overlays the piece of text. Thus, the unhealthy patient would not ordinarily be able to read the text. FIG. 17C illustrates an example of an unhealthy patient's visual field 1706 after the patient has learned an eccentric fixation point, allowing the patient to read the text displayed on the screen. For example, as shown, the patient's learned eccentric fixation point 1712 is positioned offset from the piece of text 1714. As a result, the patient may be able to read the piece of text 1714.

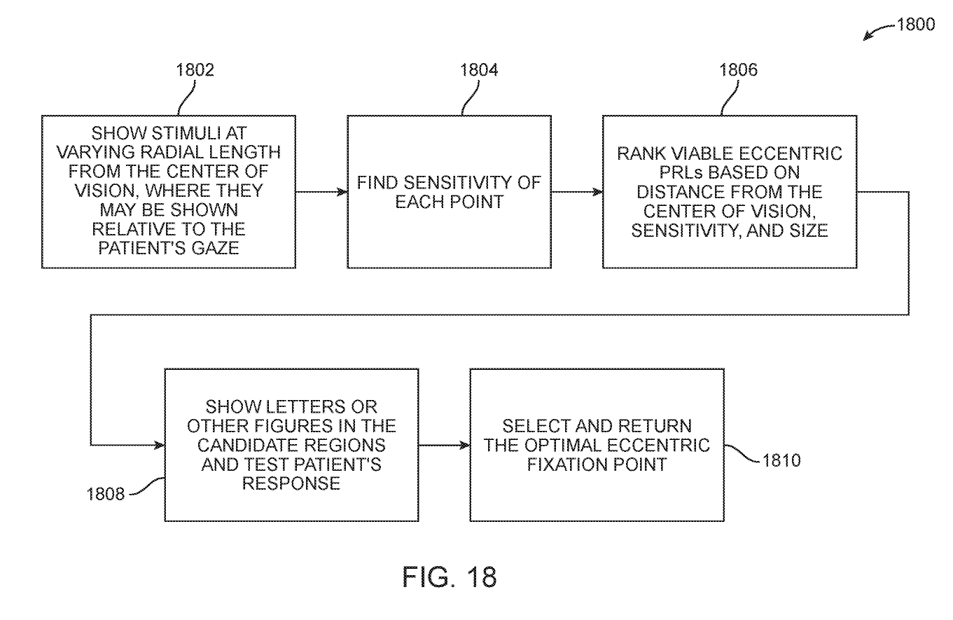

FIG. 18 illustrates an example method 1800 for performing eccentric PRL discovery on the patient, consistent with implementations of the current subject matter. In some implementations, to help a patient identify an eccentric PRL that would be most useful to the patient, the virtual reality engine 116 may display on the display screen 114A of the virtual reality headset, one or more stimuli (e.g., a series of stimuli). The one or more stimuli may include lights, colors, letters, and/or another type of visual stimuli. In response to viewing the one or more stimuli, the patient may respond verbally, or the virtual reality engine 116 may receive an input from the patient, such as via a keyboard, a clicker, an eye movement, and the like. In some implementations, the eye tracking may be implemented by the virtual reality engine 116 to monitor fixation of the patient's eye(s) and/or to display stimuli via the display screen 114A of the virtual reality headset relative to the patient's true center of vision. The virtual reality engine 116 may determine the closest point to the true center of the patient's vision that is most sensitive, based on, for example, the monitoring of the fixation of the patient's eye(s) relative to the patient's true center of vision. To determine the closest point to the true center of the patient's vision that is most sensitive, the virtual reality engine 116 may factor a distance between the fixation of the patient's eye and the natural center of vision, the patient's sensitivity to light, and the size of the sensitive area, among other factors.

For example, at 1802, the virtual reality engine 116 may display via the display screen 114A of the virtual reality headset, one or more stimuli, such as at varying radial length from the center of the patient's vision. The virtual reality engine 116 may display the one or more stimuli relative to the patient's gaze. At 1804, the virtual reality engine 116 may determine the sensitivity of the patient to each of the one or more stimuli displayed via the display screen 114A of the virtual reality headset. At 1806, the virtual reality engine 116 may rank the sensitivities and/or the viable eccentric PRLs based on one or more factors, such as the distance from the center of vision, the patient's sensitivity to each of the one or more stimuli, the size of the eccentric PRL. The virtual reality engine 116 may select one or more of the ranked viable eccentric PRLs. In some implementations, at 1808, the virtual reality engine 116 may display on the display screen 114A of the virtual reality headset, a piece of text (e.g., words, phrases, letters, images, or other figures, etc.) in the region of the one or more selected viable eccentric PRLs. The virtual reality engine 116 may test the patient's response in response to displaying the one or more viable eccentric PRLs and the piece of text. At 1810, the virtual reality engine 116 may select and return the optimal eccentric PRL (and/or fixation point).

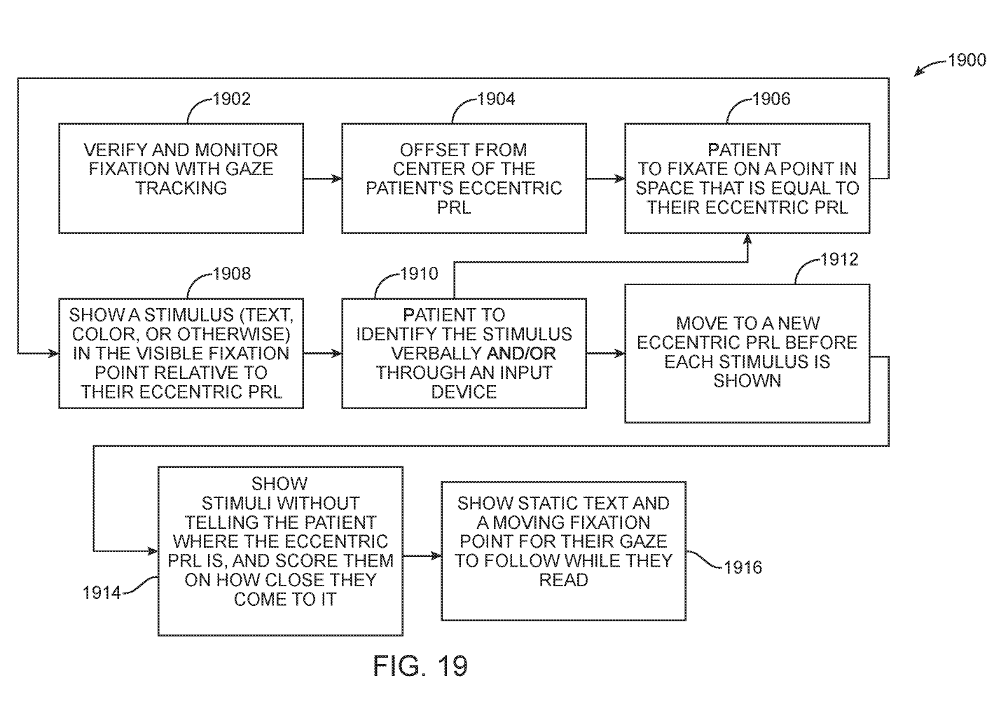

FIG. 19 illustrates an example method 1900 for performing eccentric PRL training on the patient, consistent with implementations of the current subject matter. For example, once the virtual reality engine 116 determines the optimal eccentric PRL that would allow the patient to view the piece of text, the virtual reality engine 116 may train the patient to use the optimal eccentric PRL. In some implementations, the virtual reality engine 116 may show a piece of text for the patient to read with a point to follow in order to read the text. Showing the piece of text may include a game in which important details of the game may be in the true center of the patient's vision, but the patient is guided to look at their eccentric PRL to read the text, or other training forms. The virtual reality system may use eye tracking via the virtual reality headset to determine where the patient is looking and to influence the patient to look in the correct direct (e.g., the optimal eccentric PRL), for example, by awarding the patient (awarding bonuses or other incentives), and/or deducting points from the patient. The virtual reality engine 116 may display the piece of text via the display screen 114A of the virtual reality headset. The patient may look at a certain angle and/or distance away from the displayed piece of text, at their optimal eccentric PRL) to register a successful response.

For example, at 1902, the virtual reality engine 116 may verify and/or monitor the patient's fixation with eye (or gaze) tracking. At 1904, the virtual reality engine 116 may factor in the distance the eccentric PRL is offset from the center of the patient's vision. At 1906, the virtual reality engine 116 may request that the patient fixate on a point on the display screen 114A of the virtual reality headset that is equal to the patient's eccentric PRL. At 1908, the virtual reality engine 116 may display, via the display screen 114A of the virtual reality headset, one or more stimuli (e.g., a piece of text color, and the like) in the visible fixation point relative to the patient's optimal eccentric PRL. At 1910, the patient may be asked (e.g., by the virtual reality engine) to identify the one or more stimuli (e.g., verbally and/or through an input device connected with the virtual reality headset). In some implementations, the virtual reality engine 116 may request that the patient fixate on another point on the display screen 114A of the virtual reality headset, at 1906, that is equal to the patient's eccentric PRL. In some implementations, at 1912, the virtual reality engine 116 may request that the patient focus on another eccentric PRL before each of the one or more stimuli is displayed on the display screen 114A of the virtual reality headset. At 1914, the virtual reality engine 116 may show one or more stimuli on the display screen 114A of the virtual reality headset without telling the patient where the eccentric PRL is located on the screen. The patient may be scored (e.g., by the virtual reality engine 116) based on how close the tracked patient's gaze is to the eccentric PRL. Accordingly, the virtual reality engine 116 may train the patient to use their optimal eccentric PRL, via the virtual reality headset, while performing ordinary tasks.

In some implementations, the system 100 may implement the virtual reality engine 116 and virtual reality headset to perform a visual acuity test. The virtual reality engine 116 may control the content displayed via the display screen 114A of the virtual reality headset to each of the patient's eyes. In some implementations, the virtual reality engine 116 displays text (e.g., letters) of decreasing size in one or both portions (or one or both displays) of the display screen 114A of the virtual reality headset, corresponding to each eye of the patient. The patient may be asked to identify the text, verbally (in which case the virtual reality engine 116 may recognize the patient's response using speech recognition) or via an input device described herein. Based on receipt of the patient's identification response, for example, the virtual reality engine 16 may display the next text (or letter). The virtual reality engine 116 may test each eye simultaneously, or consecutively, to produce the patient's visual acuity.

In some implementations, the administrative engine 112 may receive input from the technician to cycle between text. The technician may manually verify each patient response or input the patient's verbal responses for evaluation by the virtual reality engine 116.

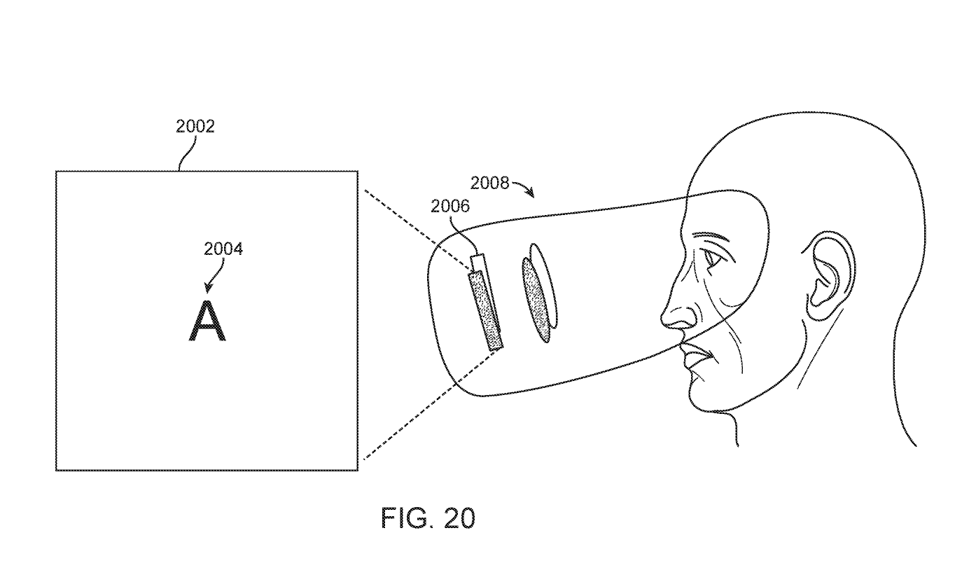

FIG. 20 illustrates an example of visual acuity testing, consistent with implementations of the current subject matter. In some implementations, the virtual reality engine 116 displays via the display screen 114A of the virtual reality headset, a visual field display 2006. The visual field display 206 may include a scene 2002 that includes a letter 2004. The letter 2004 may be positioned at an angle of the patient's total vision.

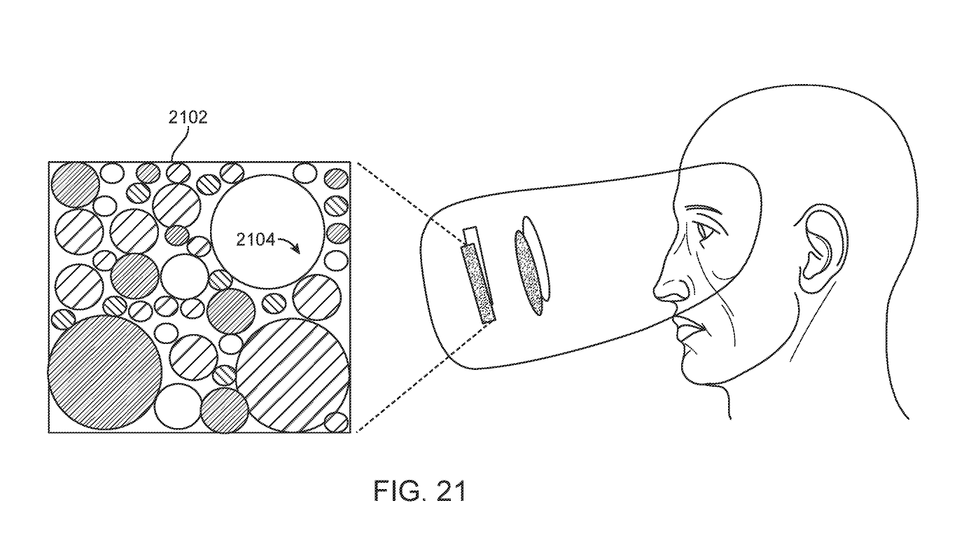

In some implementations, the system 100 may implement the virtual reality engine 116 and virtual reality headset to perform a color blindness test. For example, the virtual reality engine 116 may display via the display screen 114A of the virtual reality headset a pattern (e.g., predetermined or random) of colored circles, dots, and/or other shapes. As shown in FIG. 21, the virtual reality engine 116 may display a scene 2102 one or more circles 2104 that may have different colors to test the patient's color blindness.

### Terminology

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A virtual reality headset for performing a visual field test to detect an ocular disorder in a patient, the virtual reality headset comprising:
a display screen configured to present a test scene to the patient;
at least one data processor, the at least one data processor comprising a virtual reality engine; and
at least one memory storing instructions which, when executed by the at least one data processor, result in operations comprising:
presenting, via the display screen by the virtual reality engine, the test scene including a background color and a fixation point;
testing, automatically by the virtual reality engine via the display screen, a blind spot of the patient to thereby detect the ocular disorder, the testing including:
presenting, by the virtual reality engine on the display screen, a stimulus at a randomly assigned location from a queue of locations, the stimulus having a stimulus color; and
receiving, by the virtual reality engine, a response from the patient based on the presented stimulus.

2. The virtual reality headset of claim 1, wherein the operations further comprise: receiving, via a user interface of a display unit in communication with the virtual reality headset, one or more of patient information, the stimulus color, and the background color.

3. The virtual reality headset of claim 1, wherein the operations further comprise:
assigning, by the virtual reality engine, a timestamp to the received response from the patient; and
storing, by the virtual reality engine, in the at least one memory, the randomly assigned location of the stimulus and the assigned timestamp.

4. The virtual reality headset of claim 3, wherein the operations further comprise transmitting, by the virtual reality engine, the stored randomly assigned location of the stimulus and the assigned timestamp to a display unit in communication with the virtual reality headset.

5. The virtual reality headset of claim 1, wherein the operations further comprise: testing, automatically via the display screen, a static or kinetic perimeter of the patient, the testing including presenting the stimulus having the stimulus color on the display screen.

6. The virtual reality headset of claim 1, wherein the response from the patient comprises an invalid response.

7. The virtual reality headset of claim 6, wherein the operations further comprise comparing, by the virtual reality engine, a number of received invalid responses to a predetermined threshold; and restarting, by the virtual reality engine, the testing, when the number of received invalid responses is greater than or equal to the predetermined threshold.

8. A method of performing a visual field test to detect an ocular disorder in a patient via a virtual reality headset, the method comprising:
presenting, via a display screen by a virtual reality engine of the virtual reality headset, a test scene including a background color and a fixation point;
testing, automatically by the virtual reality engine via the display screen, a blind spot of the patient to thereby detect the ocular disorder, the testing including:
presenting, by the virtual reality engine on the display screen, a stimulus at a randomly assigned location from a queue of locations, the stimulus having a stimulus color; and
receiving, by the virtual reality engine, a response from the patient based on the presented stimulus.

9. The method of claim 8, further comprising receiving, via a user interface of a display unit in communication with the virtual reality headset, one or more of patient information, the stimulus color, and the background color.

10. The method of claim 8, further comprising:
assigning, by the virtual reality engine, a timestamp to the received response from the patient; and
storing, by the virtual reality engine, in the at least one memory, the randomly assigned location of the stimulus and the assigned timestamp.

11. The method of claim 10, further comprising transmitting, by the virtual reality engine, the stored randomly assigned location of the stimulus and the assigned timestamp to a display unit in communication with the virtual reality headset.

12. The method of claim 8, further comprising testing, automatically via the display screen, a static or kinetic perimeter of the patient, the testing including presenting the stimulus having the stimulus color on the display screen.

13. The method of claim 8, wherein the response from the patient comprises an invalid response.

14. The method of claim 13, further comprising comparing, by the virtual reality engine, a number of received invalid responses to a predetermined threshold; and restarting, by the virtual reality engine, the testing, when the number of received invalid responses is greater than or equal to the predetermined threshold.

15. A non-transitory computer-readable medium storing instructions, which when executed by at least one data processor, result in operations comprising:
presenting, via a display screen of a virtual reality headset by a virtual reality engine of the virtual reality headset, a test scene including a background color and a fixation point;
testing, automatically by the virtual reality engine via the display screen, a blind spot of a patient to thereby detect an ocular disorder in the patient, the testing including:
presenting, by the virtual reality engine on the display screen, a stimulus at a randomly assigned location from a queue of locations, the stimulus having a stimulus color; and
receiving, by the virtual reality engine, a response from the patient based on the presented stimulus.

16. The non-transitory computer-readable medium of claim 15, further comprising receiving, via a user interface of a display unit in communication with the virtual reality headset, one or more of patient information, the stimulus color, and the background color.

17. The non-transitory computer-readable medium of claim 15, wherein the operations further comprise:
assigning, by the virtual reality engine, a timestamp to the received response from the patient; and
storing, by the virtual reality engine, the randomly assigned location of the stimulus and the assigned timestamp.

18. The non-transitory computer-readable medium of claim 17, wherein the operations further comprise transmitting, by the virtual reality engine, the stored randomly assigned location of the stimulus and the assigned timestamp to a display unit in communication with the virtual reality headset.

19. The non-transitory computer-readable medium of claim 15, wherein the response from the patient comprises an invalid response.

20. The non-transitory computer-readable medium of claim 19, wherein the operations further comprise comparing, by the virtual reality engine, a number of received invalid responses to a predetermined threshold; and restarting, by the virtual reality engine, the testing, when the number of received invalid responses is greater than or equal to the predetermined threshold.
